(19) United States
(12) Patent Application Publication
(10) Pub. No.: US 2006/0105390 A1
(43) Pub. Date: May 18, 2006

(54) Method of characterizing heterogeneity in receptor-ligand interactions and related method of assay signal histogram transformation

(76) Inventors: Yi Zhang, Hillsboro, NJ (US); Michael Seul, Fanwood, NJ (US)

Correspondence Address: Eric Mirabel;Bioarray Solutions, 35 Technology Drive, Warren, NJ, 07059, US

(21) Appl. No.: 10/976,686

(22) Filed: Oct. 29, 2004

Related U.S. Application Data
(60) Provisional application No. 60/515,332, filed on Oct. 29, 2003.
(60) Provisional application No. 60/544,518, filed on Feb. 14, 2004.

(51) Int. Cl. G01N 33/53; G06F 19/00

(52) U.S. Cl. 435/7.1; 702/19

(57) ABSTRACT

Disclosed is a method for increasing the degree of confidence in receptor-ligand and probe-target interaction assays involving ligand or target capture by a heterogeneous population of receptors or probes displayed on solid phase carriers. The method comprises a process of establishing a weight function reflecting the heterogeneity in the receptor population. The weight function is applied to “filter” the assay signal distribution (“histogram”) produced in an assay involving the interaction of immobilized receptors with ligands in an actual clinical sample in order to “sharpen” the assay signal intensity distribution.

### RELATED APPLICATIONS

This application claims priority to U.S. Provisional Application No. 60/515,332, filed Oct. 29, 2003, and to No. 60/544518, filed Feb. 14, 2004.

### FIELD OF THE INVENTION

The invention relates to methods and algorithms that can be executed by a software-computer system.

### BACKGROUND

Binding assays probing the molecular interactions between receptor-ligand or probe-target pairs often involve non-uniformities in the receptor or probe population. For example, “spotting” produces highly uneven distributions as well as widely varying configurations of receptors and probes that affect diagnostic and analytical uses of “spotted arrays” of proteins and oligonucleotides (as outlined in “Multianalyte Molecular Analysis Using Application-Specific Random Particle Arrays,” U.S. application Ser. No. 10/204,799, filed on Aug. 23, 2002; WO 01/98765). Arrays of oligonucleotides or proteins also can be formed by displaying these capture moieties on chemically encoded microparticles (“beads”) which are then assembled into planar arrays composed of a set of such encoded functionalized carriers, preferably in accordance with the READ™ format (discussed in US Patent Application “Multianalyte Molecular Analysis Using Application-Specific Random Particle Arrays,” supra). Microparticle arrays displaying proteins or oligonucleotides of interest, also referred to herein for convenience collectively as receptors, can be produced by light-controlled electrokinetic assembly near semiconductor surfaces (see U.S. Pat. Nos. 6,468,811, and 6,514,771) or by a direct disposition assembly method (previously described in Provisional Application Ser. No. 60/343,621, filed Dec. 28, 2001 and in U.S. application Ser. No. 10/192,352, filed Jul. 9, 2002).

To perform protein or nucleic acid analysis, such encoded carrier arrays are placed in contact with samples anticipated to contain protein ligands or target polynucleotides of interest. Capture of ligand or target to particular capture agents displayed on carriers of corresponding type as identified by a color code produces an optical signature such as a fluorescence signal, either directly or indirectly by way of subsequent decoration, in accordance with one of several known methods (See U.S. patent application Ser. No. 10/271,602 “Multiplexed Analysis of Polymorphic Loci by Concurrent Interrogation and Enzyme-Mediated Detection,” filed Oct. 15, 2002, and Ser. No. 10/204,799 supra.), and resulting images are analyzed to extract assay signal intensity distributions. The identities of capture agents including protein receptors or oligonucleotide probes generating a positive assay signal can be determined by decoding carriers within the array.

Solid phase carriers including microparticles functionalized with either proteins or oligonucleotides can display chemical or physical heterogeneity, because, once immobilized, these molecules can adopt varying orientations or configurations so that receptor-ligand affinities display corresponding non-uniformities or heterogeneities. Further, the number of (viable or accessible) receptors per unit surface area of carrier or the number of receptors per (entire) carrier (“receptor-coverage”) can be non-uniform across a population of nominally identical carriers, for example as a result of non-uniformities or fluctuations affecting the process of receptor immobilization. These effects can introduce significant variations in assay signal intensities recorded from a population of such nominally identical, but chemically heterogeneous carriers, as described herein in several examples of receptor-ligand binding. The corresponding signal intensity distribution, also referred to herein as an intensity histogram, typically will display a significant variance and/or skew, and limit the level of statistical confidence attained in the assay.

The effect of heterogeneity on signal intensities can be seen, for example, in solid phase screening assay designed to detect allo-antibodies (“Panel-Reactive Antibodies,” or PRA) by using extracts or membrane fragments containing Class I and II antigens immobilized on encoded microparticles (see U.S. Pat. Nos. 5,948,627 and 6,150,122). FIG. 1 (from a product insert using flow cytometric analysis published by One Lambda, Inc., Canoga Park, Calif.), displays (on a logarithmic scale) a typical intensity distribution recorded in such a test. For class I as well as class II profiles, the large width of the two partially overlapping distributions representing, respectively, the negative reference and the positive control signal substantially reduces the effective discrimination attained in these assays. When positive and negative signal intensity distributions overlap, assay signals recorded from certain carriers fall into the area of overlap and cannot be designated as positive or negative. In the example of FIG. 1, the negative control produces a rather significant signal level, and the mean of the positive control exceeds that of the negative control by only a factor of approximately two. Generally, the smaller the separation between the mean of positive and negative signal distributions, and the larger the respective variances, the lower the degree of confidence in the assay results.

One aspect of heterogeneity is that arising from a distribution of affinity constants governing the receptor-ligand interaction of interest, as previously discussed (See J. V. Selinger and S. Y. Rabbany, Anal. Chem 69, 170-174 (1997); D. Lancet, E. Sadovsky, and E. Seidemann, Proc. Natl. Acad. Sci. Vol. 90, pp. 3715-3719, April 1993.) The effect of the participation of multiple receptors in ligand-binding also has been discussed (see P. J. Munson and D. Rodbard, Anal. Biochem. 107, pp. 220-239 (1980); E. M. Melikhova, I. N. Kurochkin, S. V. Zaitsev, and S. D. Varfolomeev, Anal. Biochem 175, pp. 507-515 (1988); A. K. Thakur, M. L. Jaffe, and D. Rodbard, Anal. Biochem 107, 279-295 (1980)).

However, no method has been suggested or developed to characterize a heterogeneous population in terms of a systematic parameterization based on a multiplexed “test” or “reference” assay, designed to probe that heterogeneity using suitable reference reagents such as monoclonal antibody ligands or synthetic DNA targets, and no method of analysis has been disclosed to design and apply filters in order to “sharpen” assay signal distributions, also referred to herein as histograms, to enhance the level of confidence in the assay results. A method is clearly desirable to transform assay signal histograms in a controlled and systematic way so as to improve the confidence associated with estimates of histogram moments, notably mean and variance.

### SUMMARY

Disclosed is a method for increasing the degree of confidence in receptor-ligand and probe-target interaction assays involving ligand or target capture by a heterogeneous population of receptors or probes displayed on solid phase carriers. The method comprises a process of establishing a weight function reflecting the heterogeneity in the receptor population. The weight function is applied to “filter” the assay signal distribution (“histogram”) produced in an assay involving the interaction of immobilized receptors with ligands in an actual clinical sample in order to “sharpen” the assay signal intensity distribution.

To establish a weight function, a reference assay is performed using a solution of well-defined ligand or target “test” molecules, which are permitted to interact with carrier-displayed receptors. The resulting signal intensity distribution is divided into segments representing subpopulations composed of equal numbers of carriers, and the corresponding intensity distributions are analyzed in terms of a theoretical model in order to characterize heterogeneity in the receptor population manifesting itself in the interaction of the two or more types (or states) of receptors with the test ligand; specifically, the model yields the relative density (“coverage”) of two or more distinct types (or states) of receptors within each carrier subpopulation. For the special case of a two-state model, the fractional coverage of high affinity receptors as a function of segment index represents a “heterogeneity descriptor” of the invention, also referred to herein as an η-plot. Provided that the sets of carriers used in the reference assay and in the sample assay both comprise a representative sample of the same carrier reservoir (“parent population”), the nature of receptor heterogeneity manifesting itself in the reference assay, and more specifically the η-plot, will be identical to that manifesting itself in the sample assay. Further, differences in the η-plots for the reference assay and an assay performed on an actual clinical sample provides a method of the invention of detecting heterogeneity in the ligand population. The η-plot forms the basis for the construction of a weight function by application of a mapping, as described herein.

The receptor-ligand pairs suitable for use with this method can be either protein-protein interaction pairs, including antigen-antibody and enzyme-substrate pairs, or complementary oligonucleotide pairs. The carriers can be microparticles as disclosed in U.S. application Ser. No. 10/204,799, filed on Aug. 23, 2002, and entitled: “Multianalyte Molecular Analysis Using Application-Specific Random Particle Arrays.” The methods of the present invention also can be applied to transform signals produced by other formats of solid phase molecular analysis, including signals from flow cytometry (R. Pei, G. Wang, C. Tarsitani, S. Rojo, T. Chen, S. Takemura, A. Liu, and J. Lee, Human Immun. 59, pp. 313-322 (1998)) as well as signals recorded from “spotted” arrays. The methods also can be applied to the transformation of assay signals other than optical signatures, for example, to radiation counts recorded from radioactively labeled target molecules.

The method of the present invention proceeds in several steps, the entire sequence of which is applied in turn to each carrier type. First, the distribution of mean intensities of a given type is ranked by mean intensity values and then divided into S equal subpopulations. That is, the intensity histogram, {I^{p}, N^{p}}, p counting histogram bins, is transformed into a representation of the form {k, N^{(k)}}, k ε[1, S], wherein the occupancies N^{(k)} are (initially) identical. Each subpopulation is then analyzed in terms of a two-state model, defined in terms of {K_{L}, R_{L}} and {K_{H}, R_{H}}, as well as a mixing coefficient, η; here, K_{L} and K_{H} denote the affinity constants governing the interaction of ligands or targets with receptors or probes of two types (or receptors in two states), these being present on solid phase carriers at respective numbers per carrier of R_{L} and R_{H}. These parameters are obtained by a two-pass regression analysis of adsorption isotherms for individual subpopulations. First, {K_{L}^{(k)}, R_{L}^{(k)}} and {K_{H}^{(k)}, R_{H}^{(k)}} are determined for individual subpopulations. Next, average values, K_{L}=<K_{L}^{(k)}> and K_{H}=<K_{H}^{(k)}> are calculated, and final values for the coverage parameters, R_{L}^{(k)} and R_{H}^{(k)} are obtained by again fitting adsorption isotherms for each subpopulation to the two-state model, this time keeping K_{H} and K_{L} fixed. As discussed herein, the total “coverage”, R^{(k)}=R_{L}^{(k)}+R_{H}^{(k)}, serves as a subpopulation index. To characterize the heterogeneity within the carrier population, manifesting itself in the presence of at least two types (or two distinct configurations) of receptors, one defines a parameter, η, representing the fraction of “high affinity” receptors, η=R_{H}/R; generally, as described herein, η^{(k)} will vary with R^{(k)}. The function η(R) forms the basis for construction of a weight function, {w^{k}, 1≦k≦S} which may assume several functional forms, as disclosed herein. This model produces a systematic parameterization to control the subsequent process of applying the weight function in order to “filter” the signal intensity distribution recorded in the analysis of actual samples.

To initiate the filtering operation, the intensity distribution produced in the sample assay, having been sorted by intensity, is first transformed by segmentation into S equal subpopulations, and these are mapped to corresponding subpopulations of the reference intensity histogram. Once established, this one-to-one correspondence permits the assignment of a unique weight, w^{k}, to the k-th sample histogram segment. That is, the actual filtering operation entails the multiplication of the set of occupancies, {N^{k}; 1≦k ≦S} by the weight function {w^{k}, 1≦k ≦S}. Corrected estimates of mean and variance associated with the weighted (“filtered”) subpopulations are computed in the manner described herein. To facilitate the direct comparison to the original histogram to assess the qualitative transformation of the shape of the original histogram, the filtered occupancy representation can be transformed back into an altered histogram using the procedure described herein.

The disclosed method also relates to the detection of non-uniformities in the ligand population. In principle, a reference assay would be performed using an ideal receptor to probe the heterogeneity of a representative sample of carriers. While this latter reference assay may not be available or practical in every circumstance, heterogeneity in the ligand population may be detected by analyzing the sample assay in the same way as the reference assay in order to derive a second parameterization in the form η^{k}=η(R^{k}), and comparing this to the parameterization derived from the reference assay. Any substantial difference in the parameterization of reference and sample assay manifesting itself in the construction of the respective η-plots is attributed to heterogeneity in the ligand population.

In one embodiment, signal intensity distributions are established for each type of carrier, each such type corresponding to one or more types of receptors or probes, by evaluating the mean intensity of all carriers of a given type. For this embodiment, the method averages over spatial non-uniformities occurring on individual carriers in a manner corresponding, for example, to flow cytometric analysis of microparticle-displayed binding complexes. The Random Encoded Array Detection (READ) format, a preferred embodiment of multianalyte molecular analysis, permits the extension of the methods of the present invention to smaller length scales, as discussed herein.

These methods are described further below with reference to the drawings.

### BRIEF DESCRIPTION OF THE DRAWINGS

FIG. 1 shows the signal intensity distribution recorded by flow cytometry in an assay monitoring the reaction between human allo-serum with specificity to HLA Class I (hAb-I) (FIG. 1A) and human allo-serum with specificity to HLA Class II (hAb-I) (FIG. 1B), respectively, and HLA Class I and HLA Class II antigens extracted from cell membranes and immobilized on microparticles (“beads”) (See product insert for flow cytometric analysis published by One Lambda, Inc., Canoga Park, Calif.);

FIG. 2 shows assay signal intensity distributions recorded in the READ™ format for a reaction between human allo-serum with specificity to HLA Class II (hAb-II), at four serum dilution ratios (FIGS. 2A-2D), and HLA Class II antigens extracted from cell membranes and immobilized on microparticles (“beads”) and further shows the dependence of the carrier mean intensity and corresponding variance of the assay signals on dilution ratio (FIG. 2E).

FIG. 3 shows assay signal intensity distributions recorded in the READ™ format for a reaction between a solution containing fluorescently labeled synthetic 90-mer oligonucleotide, at four different concentrations (FIGS. 3A-D), and a matching 25-mer oligonucleotide probe covalently attached to microparticles.

FIG. 4 shows the dependence of carrier mean intensity and corresponding variance of assay signal intensity distributions, recorded in the READ™ format for the reaction between anti- HLA Class I monoclonal antibody (mAb-I) and HLA Class I and HLA Class II antigens extracted from cell membranes as a function of target mAb-I antibody concentration.

FIG. 5 shows the dependence of carrier mean intensity and corresponding variance of assay signal intensity distributions, recorded in the READ™ format for the reaction between anti- HLA Class II monoclonal antibody (mAb-II) and HLA Class I and HLA Class II antigens extracted from cell membranes as a function of target mAb-II antibody concentration.

FIG. 6 shows assay signal intensity distributions recorded in the READ™ format for a reaction between anti-HLA Class II monoclonal antibody (mAb-II), at four concentrations (FIGS. 5A-5D), and HLA Class II antigens extracted from cell membranes and immobilized on microparticles (“beads”).

FIG. 7 shows the dependence of the ratio of “B”, the mean value of all carrier mean intensities, indicating the total number of mAb-I antibodies “bound“to HLA Class I antigens extracted from cell membranes and immobilized on microparticles and “F”, the concentration of “free” mAb-I antibody, as a function of B. The solid line represents a fit to the two-state model disclosed herein, producing the optimal values of the binding parameters, K_{H}, R_{H}, K_{L}, R_{L} and η, shown in the inset.

FIG. 8 shows the dependence of the ratio of “B”, the mean value of all carrier mean intensities, indicating the total number of mAb-II antibodies “bound” to HLA Class II antigens extracted from cell membranes and immobilized on microparticles and “F”, the concentration of “free” mAb-II antibody, as a function of B. The solid line represents a fit to the two-state model disclosed herein, producing the optimal values of the binding parameters, K_{H}, R_{H}, K_{L}, R_{L} and η, shown in the inset.

FIG. 9 shows the dependence of the ratio of “B”, the mean value of all carrier mean intensities, indicating the total number of anti-Jo-1 antibodies “bound” to affinity-purified Jo-1 antigens immobilized on microparticles and “F”, the concentration of “free” anti-Jo-1 antibody, as a function of B. The solid line represents a fit to the two-state model disclosed herein, producing the optimal values of the binding parameters, K_{H}, R_{H}, K_{L}, R_{L} and η, shown in the inset.

FIG. 10A shows the dependence of the ratio of “B”, the mean value of all carrier mean intensities, indicating the total number of mAb-II antibodies “bound” to HLA Class II antigens extracted from cell membranes and immobilized on microparticles and ”F”, the concentration of “free” mAb-II antibody, as a function of B (see also FIG. 7), for five quintiles of the original distribution (FIG. 2, see also: FIG. 7), denoted by Q1 to Q5. l The dotted lines represent straight line approximations to the asymptotic regimes, and the solid lines represent fits to the two-state model disclosed herein, producing the set of optimal values of the binding parameters, K_{H}, K_{L}, R and η, shown in the inset.

FIG. 10B: the solid line connecting the intersection points of the dotted lines represents the crossover locus; the inset shows the dependence of the parameter η on R, as determined from the regression analysis producing the fits shown as solid lines.

FIG. 10C illustrates the segmentation of intensity distributions into subpopulations differing in coverage and illustrates the mapping of corresponding segments of intensity distributions produced in different assays. FIG. 10E shows a discrete function η^{k}(R^{k}) constructed by using optimal parameters obtained from regression analysis of segments using a two-state model and compares it to the underlying continuous function η^{(R)}. FIG. 10F shows two actual discrete functions, η^{k}(R^{k}), obtained by analysis of the reference assay using mAb-II, with S=5 and S=10.

FIG. 10D: the solid (dotted) line represents the predicted effective affinity constant of the asymptotes of the high (low)-affinity branch computed using fitted binding parameters.

FIG. 11 shows the set of fits of FIG. 10A, for quintiles Q1 to Q5, reproduced here in terms of the notation of the theoretical description given in the disclosure, showing the dependence of the ratio of occupancy fraction, ξ^{k}, to ligand concentration, Cₗ, as a function of ξ^{k}. The three straight lines marked “Cₗ high”, “Cₗ medium” and “Cₗ low”, represent loci of constant initial ligand concentration. Also illustrated, in the form of projections of the isotherms on the ordinate, are expected shapes of signal intensity profiles.

FIG. 12 shows the sequence of transforming, filtering and back transforming a given assay signal distribution (“histogram”), illustrated here for a segmentation of the original histogram into five subpopulations, all having identical areas.

FIG. 13 shows a parameterization, η=η(R^{k}) for the data in FIG. 10, here using S=10 (FIG. 13A), a linear mapping function, w=w(η) (FIG. 13B), and a corresponding weight function, w=w(k) (FIG. 13C); the weight function coefficients are normalized such that

$${\sum }_{k} {w}_{k} = 1.$$

FIG. 14, 15, 16 and 17 show examples of histogram transformations on assay intensity data recorded for reaction between hAb-II positive serum and HLA Class II antigens at immobilized on encoded microparticles, at four levels of dilution, as indicated in each panel (see details in Example 2), produced by filtering using a weight functions derived by application of a linear mapping function; the respective upper panels show the original histogram (top) and an intermediate representation of a filtered histogram having S bins (bottom); the respective lower panels show the original histogram (top) and a representation of the filtered histogram using a large number of bins of equal size.

FIG. 18 shows a comparison of the filtered histograms produced from the original histogram in FIG. 16—reproduced here in the top panels of FIGS. 18A and 18B—by application of a linear and a non-linear mapping functions, shown in the respective insets.

FIG. 19A shows a graphical illustration relating to the application of the methods of the invention to the analysis of intensity distributions recorded from a “spotted” cDNA array. FIG. 19A shows the division of a spot of the array into a number of “tiles”, representing entities analogous to “carriers”, a terminology adopted in the disclosed invention. FIG. 19B illustrates the segmentation and transformation applied to histogram of tile mean intensities by applying the disclosed methodology. FIG. 19C illustrates an alternate way of dividing a cDNA spot by reading intensities through a mask with small apertures at random positions.

### DETAILED DESCRIPTION

1. Characterizing Heterogeneity in Receptor-Ligand Interactions

Interactions between ligands and their cognate receptors in a solid phase assay format frequently produce signals of considerable variance indicating chemical heterogeneity in the receptor or ligand populations. Thus, it has long been recognized in the art that the interaction between ligand proteins (used herein to include target nucleic acid) in solution and immobilized receptor proteins (used herein to include probe nucleic acids) will be affected by the physical-chemical characteristics of the solid phase (Hermanson, G. T. “Bioconjugate Techniques” Academic Press, San Diego, Calif., 1996; J. E. Butler “Solid Phases in Immunoassay” pp 205-225 in Immunoassay, Eds. Diamandis, E. P., Christopoulos, T. K. Academic Press, San Diego, Calif., 1996). For example, when receptors are immobilized on nylon membranes or other planar substrates, local variations in surface topography or surface chemical characteristics including the lateral density of “spotted” receptors will affect the assay signal intensity. When receptors are immobilized on microparticles (“beads”), the physical-chemical characteristics of a population of such carriers and their effects on the receptor population will affect the assay signal distribution produced by that receptor population. Immobilized receptors, as in the case of proteins may exist in multiple configurations of varying viability in which epitopes are occluded or denatured or, as in the case of nucleic acid probes, may assume a wide variety of configurations determined by random adsorption to the solid phase, limiting the accessibility of cognate subsequences.

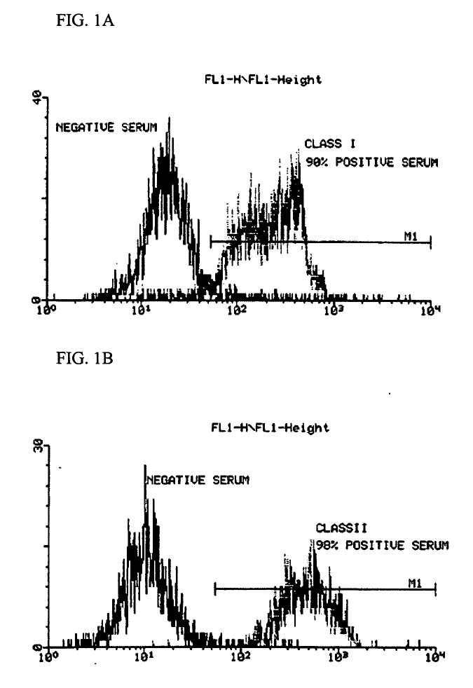

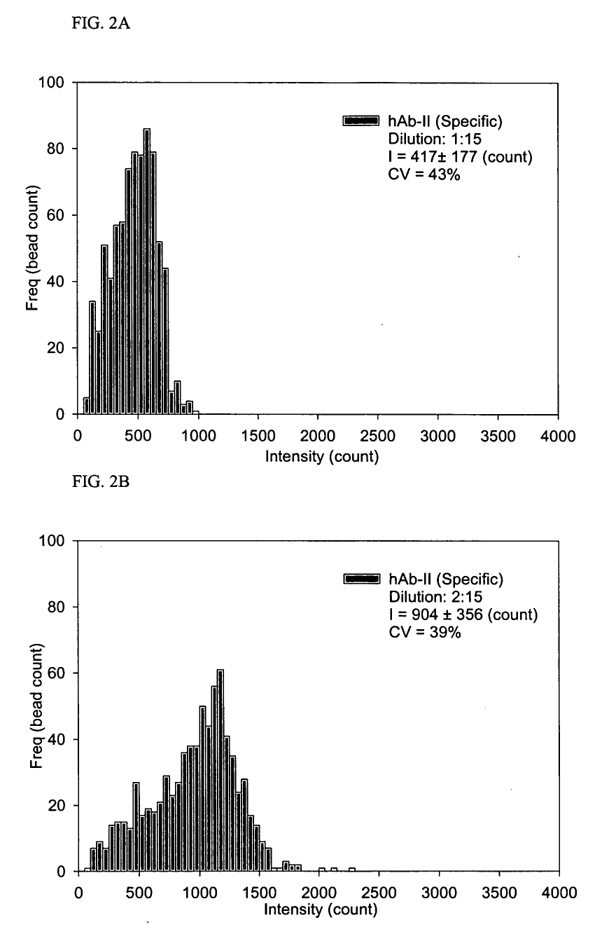

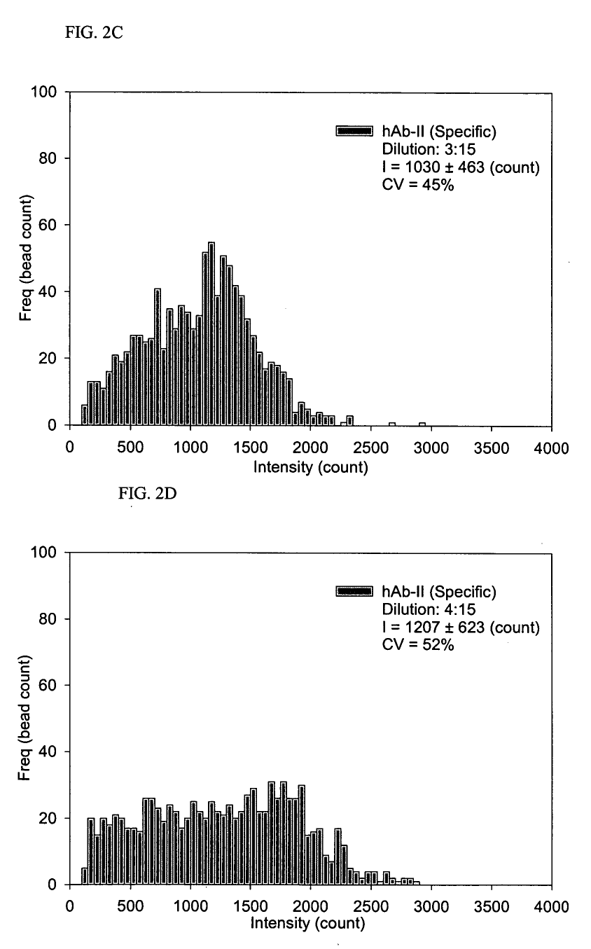

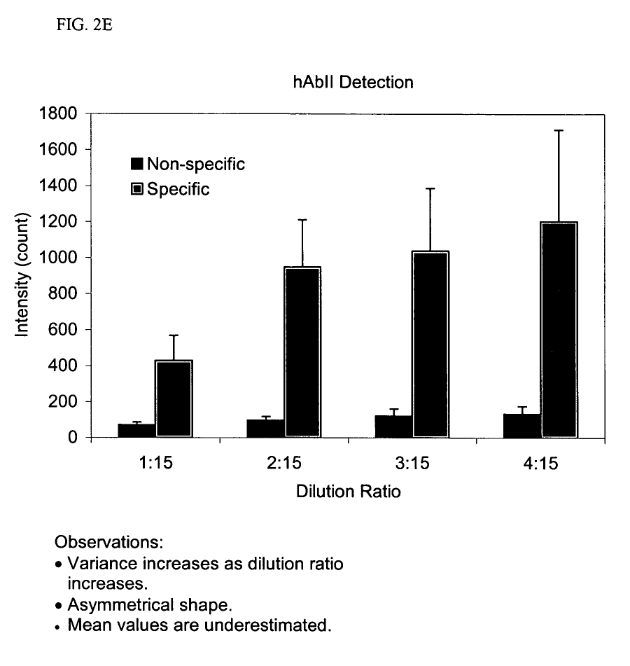

This phenomenon is illustrated here by the example of an allo-antibody (also referred to as “Panel-Reactive Antibody”, or PRA) profiling assay performed using extracts of fragments of cellular membranes affixed to encoded microparticles (“beads”), each bead type representing a small number (“pool”) of human leukocyte antigens (HLA) (see FIG. 1). Allo-antibody profiling, performed in the Random Encoded Array Detection (READ™) format of multi-analyte molecular analysis as described in Examples 1 and 2. Assay signal intensity histograms for the reaction of human class II serum (“hAb-II”) at several dilutions with class II antigens extracted from the membranes of certain cell lines display a significant variance (CV ˜40%) and significant skew toward low intensity. Signal intensity histograms produced in an assay performed as described in Example 3 to determine the level of anti-Jo-1, an auto-antibody associated with poliomyositis, by permitting patient sera to contact an array of microparticles functionalized with affinity-purified Jo-1 antigen, display similar characteristics (not shown). Simply extracting the mean of such a broad distribution, shown along with the variance for different hAb-II serum dilutions in FIG. 2E, may seriously underestimate the “true” signal mean. In a theoretical description of the present invention, these effects are attributed to heterogeneity in receptor and ligand populations.

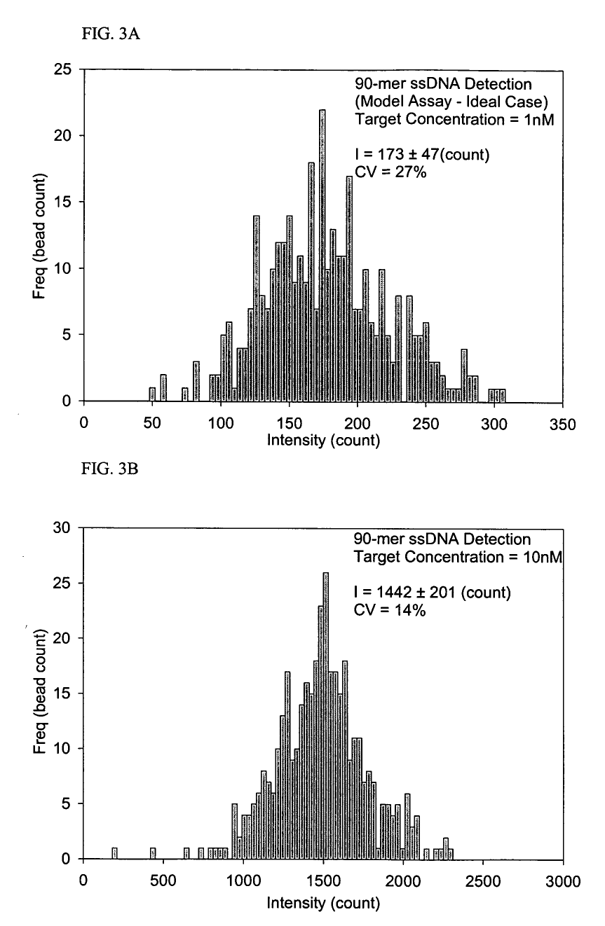

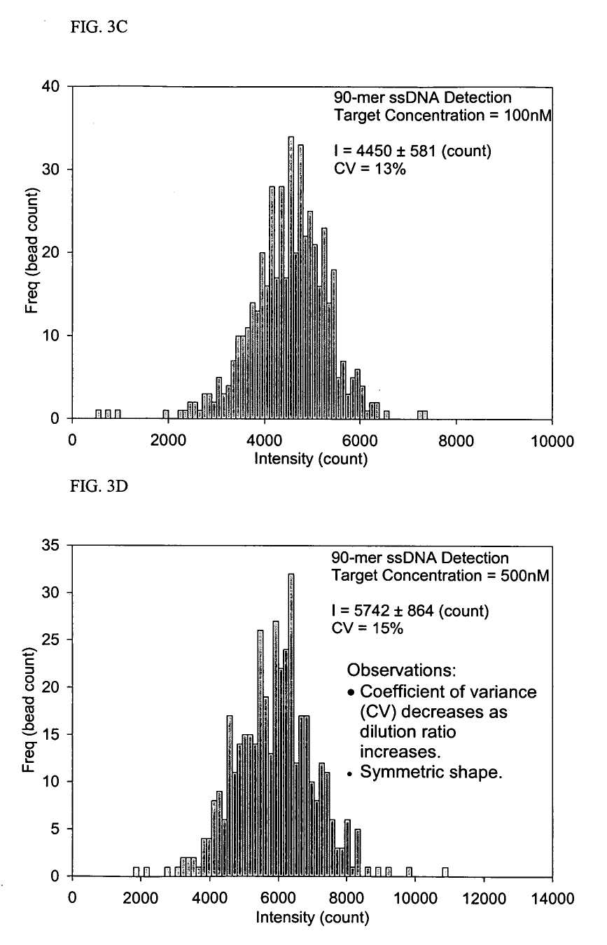

In contrast, as illustrated here (FIG. 3) by a probe-target hybridization assay involving the capture of a fluorescently labeled, synthetic 90-mer nucleic acid target strand by a 25-mer probe covalently attached to encoded microparticles, assay signal distributions display a symmetric shape and a variance which decreases as the mean increases. The variance observed in this “model” assay reflects statistical fluctuations in physical-chemical properties of the microparticle population such as the variation in diameter—typically ˜3% for the microparticles used in the assays described in the Examples given herein—and the corresponding variation in surface area, leading to a corresponding variation in total receptor coverage and total intensity per particle.

However, the asymmetric shape and significant variance of the signal intensity histograms, and the fact that both features increase with increasing ligand concentration, is not accounted for by the usual statistical fluctuations of physical-chemical characteristics of the carrier population, pointing instead to heterogeneities in the receptor and/or ligand populations. The READ-PRA assay provides direct evidence of heterogeneity in the form of images recorded from arrays of beads in the saturation regime of the titration curves in which ligands occupy all available receptors. For example, assay images (not shown) of the fluorescence signals produced by decoration (using a Cy5 dye-labeled secondary antibodies) of hAb-II captured to bead-displayed class II antigens reveal spatial non-uniformities in the signal intensity across individual beads.

These observations suggest that a likely explanation for the observed characteristics of the signal intensity distribution lies in the heterogeneity of the population of immobilized receptors.

It will be desirable to compensate for such heterogeneity in a systematic manner, and the present invention discloses, for this purpose, a systematic procedure that identifies populations of low-affinity and high-affinity receptors within carrier subpopulations and places relatively lower weight on the signal contribution from low-affinity receptors (or carriers displaying them), and higher weight on high-affinity receptors (or carriers displaying them), thereby producing a “filtered” distribution. The invention thus provides, first, a method of characterizing the heterogeneity in receptor-ligand (used herein to include nucleic acid probe-target) interactions manifesting itself in characteristic fashion, as illustrated above, in the shape of signal intensity distributions, and further, provides a method of “sharpening” such distributions in order to enhance the reliability of the assay results.

1-1. Characterizing Heterogeneity in Receptor Population

The characterization method of the invention employs a homogeneous population of well-defined ligand “test” molecules to probe a heterogeneous receptor population displayed on microparticles or other solid phase carriers. In this “reference assay,” an adsorption isotherm is obtained by varying the test ligand concentration and recording the effect of this variation on the signal intensity distribution. The adsorption isotherm is analyzed in terms of a two-state model, described herein below, to extract binding parameters, namely, affinity constants and values of receptor coverage, to parameterize the heterogeneity of the receptor population. This process is akin to that of determining the point spread function of an optical instrument, or more generally, the transfer function of a linear system. As with the finite resolution of linear systems, the heterogeneity of a receptor population manifests itself, in the form of a convolution, in any measurement performed with that receptor population.

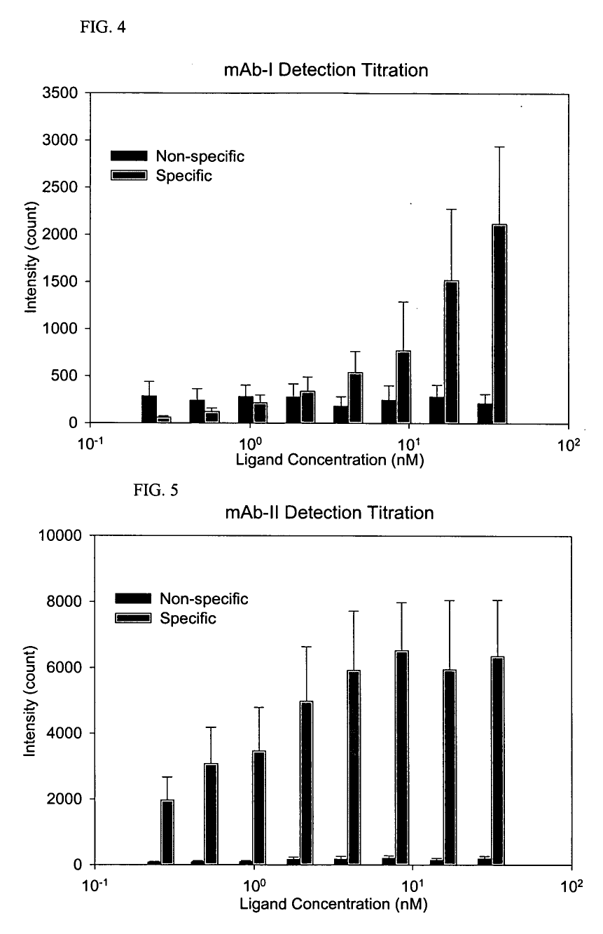

Specifically, to characterize the heterogeneity underlying the results described in connection with FIG. 2, mouse monoclonal antibodies specific to HLA Class I and Class II (abbreviated herein as mAb-I and mAb-II) were used as “test” ligands to probe the population of human HLA Class I and Class II affixed to encoded beads. FIGS. 4 and 5, respectively, show the evolution of intensity mean and intensity variance as a function of mAb-I and mAb-II concentration. FIG. 5 shows a set of assay signal intensity histograms recorded from one type of class II carriers exposed to solutions containing increasing amounts of the mAb-II test ligand. As with the distributions produced by the reaction of hAb-II serum and class II antigen (FIG. 2), the histogram peaks are broad and skewed, particularly at low ligand concentration.

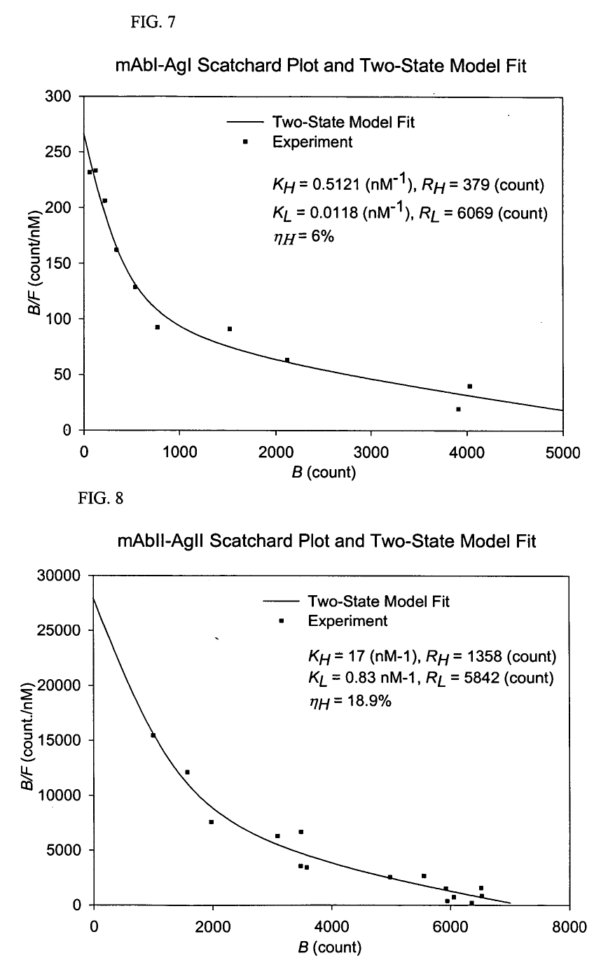

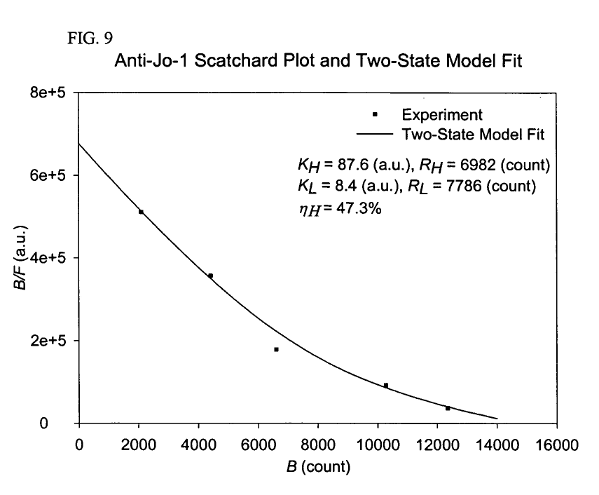

FIGS. 7 and 8 respectively show titration curves for the mAb-I and mAb-II reference assays after conversion into a representation also known as a Scatchard plot. In these plots, the abscissa represents the mean value of the mean intensities, \overline{I}, computed over all carriers of a given type, which is presumed or known to be proportional to the number of bound antibody molecules, and the ordinate represents the ratio of \overline{I} to the unbound (or free) antibody concentration. In this representation, a homogeneous receptor population, characterized by a single affinity constant and uniform and identical receptor coverage among all carriers, will produce a straight line with a negative slope, of which the absolute value is the affinity constant, and with an x-axis intercept yielding the receptor coverage. In contrast, the adsorption isotherms for the class I and class II READ-PRA assays in FIGS. 7 and 8 as well as the adsorption isotherms for the anti-Jo1 profiling assay (FIG. 9), indicate the existence of at least two regimes, differing in both affinity constant and receptor coverage.

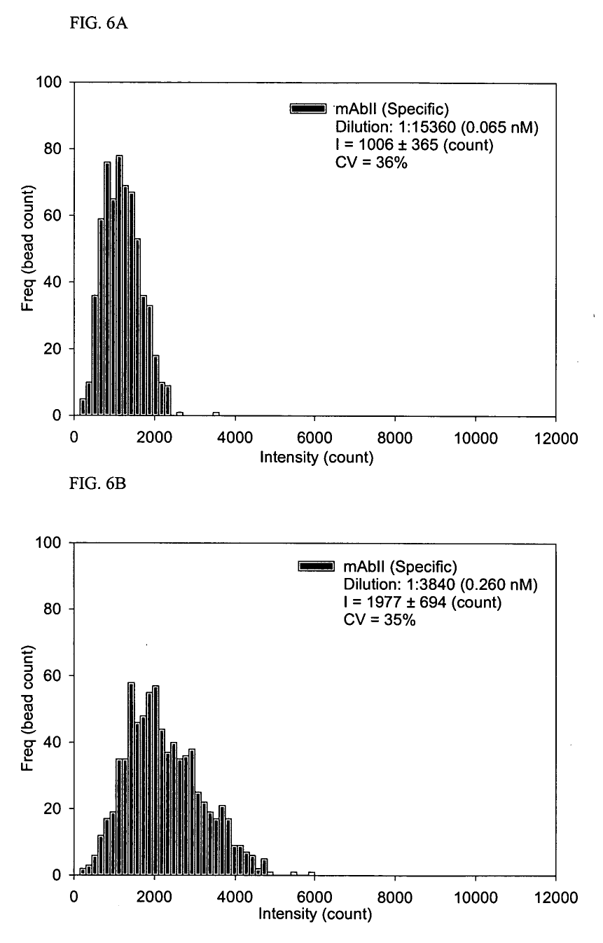

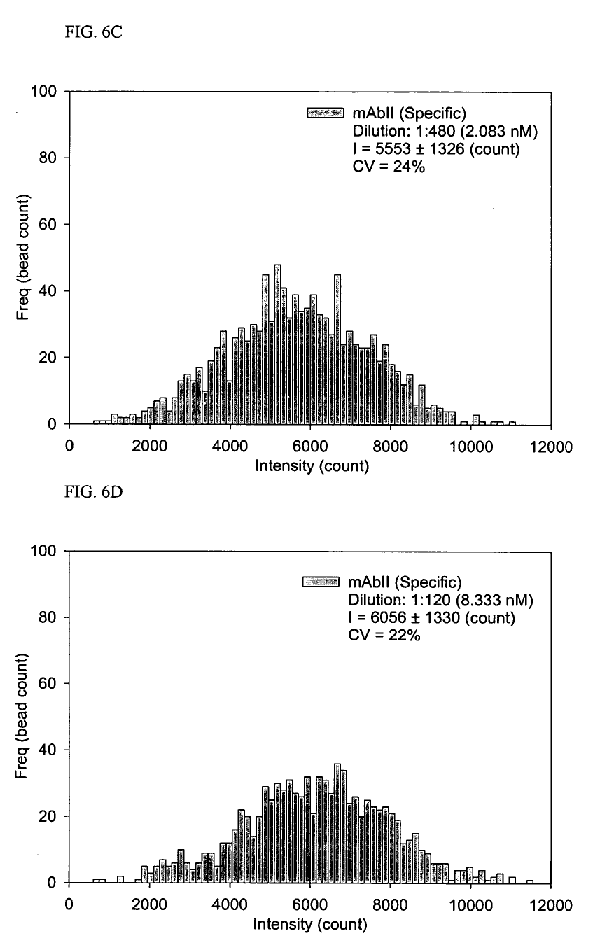

While this conclusion is confirmed by regression analysis of the adsorption isotherms in terms of the two-state model of the invention which yields the fits shown as solid lines in FIGS. 6-8 as described presently, the existence of at least two regimes for each subpopulation is readily apparent without regression analysis by simply constructing straight line approximations to the asymptotes in both regimes. One regime, corresponding to the upper portion of the plots in FIGS. 6-8, is characterized by a larger slope, hence higher affinity, and lower (extrapolated) x-intercept, hence lower value of receptor coverage, while the other regime, corresponding to the lower portion of the plots, is characterized by a smaller slope, hence lower affinity, and higher (extrapolated) x-intercept, hence higher value of receptor coverage. If one denotes the values of the affinity constants associated with high (and low)-affinity receptors as K_{H} (and K_{L}), respectively, and the values of coverage as R_{H} (and R_{L}), respectively, it will be convenient to parameterize the above features in the Scatchard plots in terms of a mixing coefficient, η≡R_{H}/R where R≡R_{H}+R_{L}.

1-2 Parameterization and Analysis of Assay Intensity Signal Distributions

Introduced is a method of parametrizing the heterogeneity of a receptor population, manifesting itself in the form of at least two branches in the adsorption isotherms, for example in the Scatchard (or analogous) representation and indicating the existence of receptors of differing affinity and differing values of receptor coverage within subpopulations of nominally identical solid phase carriers such as microparticles. This method of the invention of characterizing the heterogeneity in a receptor population proceeds by dividing the signal intensity histogram for an entire carrier population (of given type), sorted by mean intensity values, into S equal subpopulations, and establishing the form of the dependence of η^{k}≡R_{H}^{k}/R^{k} on R^{k}, 1≦k≦S. This parameterization serves as a quantitative “descriptor” of a carrier population displaying receptor(s) of interest, and also forms the basis for a filtering operation, described in detail in Sect. 2.

1-2-1. Scatchard Analysis: Multiple Receptor Populations

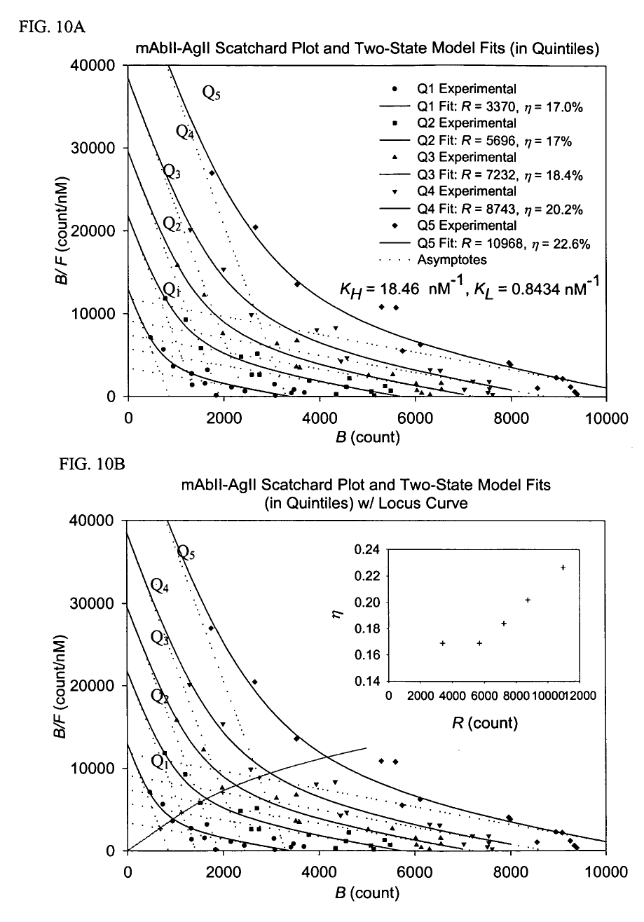

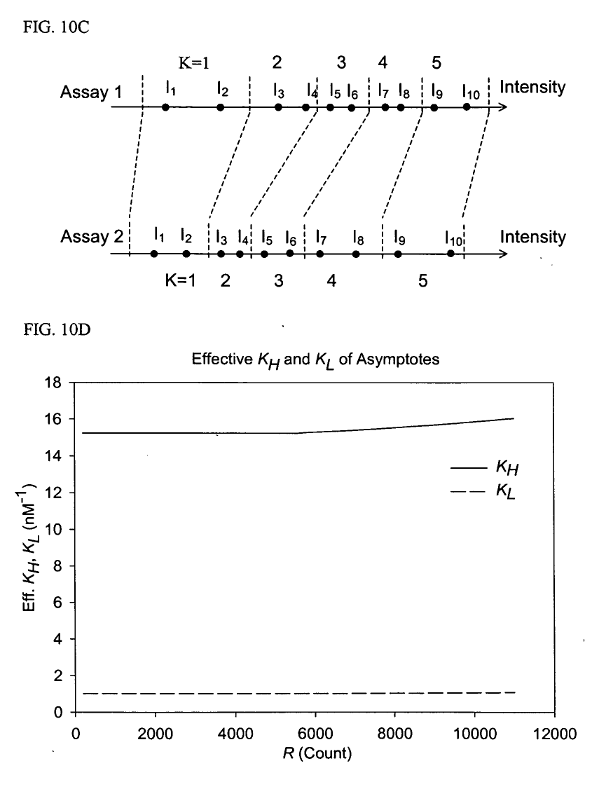

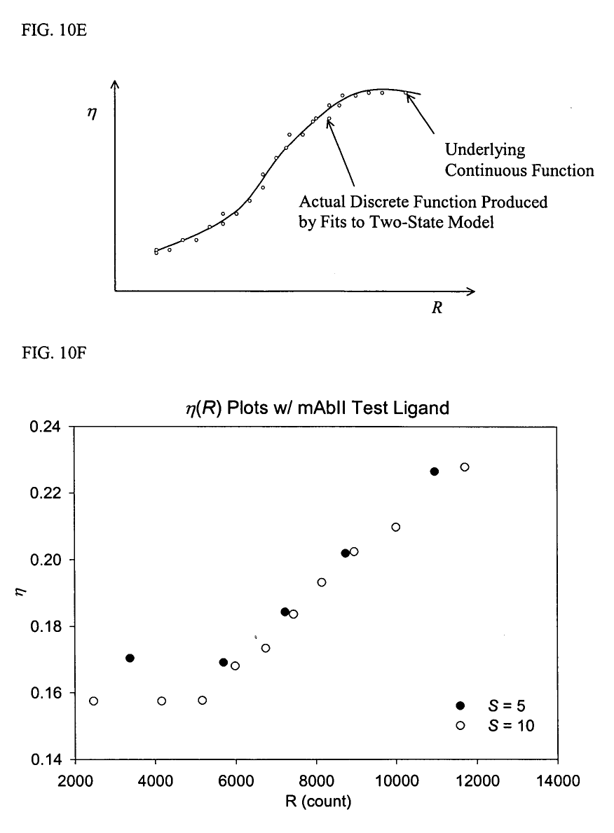

To establish the functional dependence of η^{k} on the parameter R^{k}, intensities recorded in the reference assay (FIG. 8) are sorted and then segmented into S subpopulations. For example, in the case of mAb-II isotherm, the intensity distribution was segmented into quintiles (S=5). Adsorption isotherms for each subpopulation, analyzed by applying the straight-line approximations to the high-affinity and low-affinity branches of the adsorption isotherms for each subpopulation (FIG. 10A), indicate similar values of K_{H} or K_{L} for all subpopulations, as well as widely varying values of coverage, R_{H}^{k} and R_{L}^{k}. The corresponding η^{k} (more precisely computed later), as shown in the FIG. 10B (inset), is seen to increase with increasing R^{k}, suggesting that a higher fraction of high affinity receptors is associated with higher (total) coverage. As elaborated in connection with the presentation of a theoretical framework, such dependence implies that the shape of the intensity distribution will change as ligand concentration is varied. The function η^{k}(R^{k}), as an approximation of the underlying function ηⁱ(Rⁱ) that is derived within the theoretical framework given herein, reflects the heterogeneity of bead population itself and thus can be utilized to construct a filter to suppress the contribution from low-affinity receptors to the overall assay signal.

Since adsorption isotherms for the different carrier subpopulations display similar slopes for asymptotes of high-affinity and low-affinity regimes and do not cross, as evident in FIGS. 10A and 10B, k and R^{k} have the same ordering, and one can thus use R^{k} as an alternate index to k to denote the different subpopulations that differ in total receptor coverage. Further, if the aliquot of carriers chosen for the experiments producing the data in FIG. 10A is a representative sample of the carrier parent population, the set of values {R^{k}} is intrinsic to that parent population. Segments of two intensity histograms produced in assays using aliquots of carriers drawn from the same reservoir will exhibit a one-to-one correspondence.

In practice, only intensity values are sorted and segmented, and values of receptor coverage are obtained by regression analysis to confirm same ordering. As illustrated in FIG. 10C, although a first assay (“Assay-1”) and a second assay (“Assay-2”), performed on a given sample using different ligand concentrations, may produce different intensity histograms, the one-to-one correspondence between k-th segments in the first and the second histograms will ensure that corresponding carrier subpopulations are properly identified.

1-2-2. Theoretical Description

The theoretical description, while presented here for the case of averaging carrier-internal non-uniformities to produce distributions of carrier mean intensities, is readily extended to smaller (“sub-carrier”) length scales, as discussed herein below.
The assumptions underlying this analysis are as follows:
   1. The receptor-ligand interaction is governed by the law of mass action;
   2. Several types of receptors, indexed by j, are present on their host carriers. Carriers differ from each other by the number of displaying receptors (“or coverage”), denoted by Rᵢ, with index i representing subpopulations that can be, in practice, differentiated by means of ranking and segmentation of intensity histogram;
   3. The receptor-ligand binding reaction is univalent, and receptor-ligand binding events are uncorrelated to each other; then:
   4. The mean intensity of any subpopulation of carriers is proportional to the number of captured (bound) labeled ligand molecules on such carriers following all necessary signal preprocessing.
      The following quantities and variables are defined as follows:
Cₗᵣ—Number of receptor-ligand complexes (number of bound ligands) per carrier
Cₗᵣⁱ—Number of receptor-ligand complexes (number of bound ligands) per carrier of i^{th} coverage
Cₗᵣ^{ij}—Number of receptor-ligand complexes (number of bound ligands) per carrier of j^{th} receptor type and i^{th} coverage.
η^{ij}≡Cᵣ₀^{ij}/Cᵣ₀ⁱ—Fraction of receptors of type j on carrier of i^{th} coverage; note: Σⱼη^{ij}=1.
Cₗ—Concentration of unbound ligand in liquid phase.
Cᵣ—Number of unoccupied receptors per carrier
Cᵣⁱ—Number of unoccupied receptors per carrier of i^{th} coverage.
Cᵣ₀ⁱ—Total number of receptors per carrier of i^{th} coverage.
Cᵣ₀^{ij}—Total number of receptors per carrier of j^{th} type and i^{th} coverage
Nⁱ—Number of carriers in subpopulation of i^{th} coverage.
The distribution of the set {Cₗᵣ₀ⁱ} is presorted in ascending order, i.e., Cₗᵣ₀^{m}≦Cₗᵣ₀ⁿ,∀m<n.
The law of mass action states that, the affinity, K^{j}, of the j-th receptor type is determined by the expression

$$\frac{{C}_{lr}^{i , j}}{( {C}_{r 0}^{i , j} - {C}_{lr}^{i , j} ) {C}_{l}} = {K}^{j} .$$

For each carrier of i-th coverage, the number of receptor-ligand complexes on each carrier can be expressed as:

$$\frac{{C}_{lr}^{i}}{{C}_{l}} = {\sum }_{j} \frac{{C}_{lr}^{i , j}}{{C}_{l}} = {\sum }_{j} \frac{{K}^{j} {C}_{r 0}^{i , j}}{1 + {K}^{j} {C}_{l}} = {C}_{r 0}^{i} {\sum }_{j} \frac{{K}^{j} {η}^{i , j}}{1 + {K}^{j} {C}_{l}} .$$

The mean intensity average over all coverage is thus given by,

$$\frac{{C}_{lr}}{{C}_{l}} = 〈 \frac{{C}_{lr}^{i}}{{C}_{l}} 〉 = \frac{1}{{\sum }_{i} {N}^{ i}} {\sum }_{i} {N}^{ i} {C}_{r 0}^{i} {\sum }_{j} \frac{{K}^{j} {η}^{ i , j}}{1 + {K}^{j} {C}_{l}}$$

For a continuous affinity distribution, the corresponding expression is:

$$\frac{{C}_{lr}}{{C}_{l}} = 〈 \frac{{C}_{lr}^{i}}{{C}_{l}} 〉 = \frac{1}{{\sum }_{i} {N}^{ i}} {\sum }_{i} {N}^{ i} {C}_{r 0}^{i} {\int }_{0}^{∞} ⅆ K \frac{K {ρ}^{i} ( K )}{1 + K {C}_{l}} ,$$

where ρⁱ(K) is the probability density of the affinity, K, of the i^{th} coverage.

When two receptor types with differing affinity constants participate in the binding interaction, or, when a single receptor type exists in two discrete affinity states, this model is termed herein a “two-receptor model,” or a “two-state model”. The following notations apply to a two-receptor/state model:
K_{H}—Affinity constant of high-affinity-type/state.
K_{L}—Affinity constant of low-affinity-type/state.
ηⁱ_{H}—Fraction of all high-affinity receptors on each carrier of i^{th} coverage.
Rⁱ_{H}≡C^{i,H}ᵣₒ=ηⁱ_{H}Cⁱᵣ₀—Number of all high-affinity receptors on each carrier of i^{th} coverage.
Rⁱ_{L}≡C^{i,L}ᵣₒ=(1−ηⁱ_{H})Cⁱᵣₒ—Number of all low-affinity receptors on each carrier of i^{th} coverage.
Rⁱ≡Rⁱ_{H}+Rⁱ_{L}=Cⁱᵣₒ—Same as Cᵣ₀ⁱ.
For each carrier of the i^{th} coverage, the number of receptor-ligand complexes can be expressed as:

$$\frac{{C}_{lr}^{i}}{{C}_{l}} = {C}_{ro}^{i} [ \frac{{K}_{H}}{1 + {K}_{H} {C}_{l}} {η}_{H}^{ i} + \frac{{K}_{L}}{1 + {K}_{L} {C}_{l}} ( 1 - {η}_{H}^{i} ) ] .$$

Solving for the quantity Cₗᵣⁱ/Cₗ, one obtains:

$$\frac{{C}_{lr}^{i}}{{C}_{l}} = \frac{1}{2} \{ \begin{matrix}[ {K}_{H} ( {η}_{H}^{i} {C}_{r 0}^{i} - {C}_{lr}^{i} ) + {K}_{L} ( ( 1 - {η}_{H}^{ i} ) {C}_{ro}^{ i} - {C}_{lr}^{i} ) ] + \\ \{ {[ {K}_{H} ( {η}_{H}^{ i} {C}_{r 0}^{i} - {C}_{lr}^{i} ) + {K}_{L} ( ( 1 - {η}_{H}^{ i} ) {C}_{ro}^{i} - {C}_{lr}^{i} ) ]}^{2} - \\ {4 {K}_{H} {K}_{L} {C}_{lr}^{i} ( {C}_{lr}^{i} - {C}_{ro}^{i} ) \}}^{\frac{1}{2}}\end{matrix} \}$$

If one defines

$${ξ}^{i} ≡ \frac{{C}_{lr}^{ i}}{{C}_{ro}^{i}}$$

as the occupancy fraction of receptors on each carrier of the i^{th} coverage, the two-state/receptor model yields:

$${ξ}^{ i} ( \frac{{C}_{r 0}^{i}}{{C}_{l}} ) = \frac{1}{2} {C}_{r 0}^{ i} \{ \begin{matrix}[ {K}_{H} ( {η}_{H}^{i} - {ξ}^{ i} ) + {K}_{L} ( 1 - {η}_{H}^{ i} - {ξ}^{i} ) ] + \\ \{ {[ {K}_{H} ( {η}_{H}^{ i} - {ξ}^{ i} ) + {K}_{L} ( 1 - {η}_{H}^{ i} - {ξ}^{ i} ) ]}^{2} - \\ {4 {K}_{H} {K}_{L} {ξ}^{ i} ( 1 - {ξ}^{ i} ) \}}^{\frac{1}{2}}\end{matrix} \}$$

Canceling Cᵣ₀ⁱ from both sides, one then derives ξⁱ/Cₗ and thus ξⁱ as:

$$\frac{{ξ}^{ i}}{{C}_{l}} = \frac{1}{2} \{ \begin{matrix}[ {K}_{H} ( {η}_{H}^{i} - {ξ}^{ i} ) + {K}_{L} ( 1 - {η}_{H}^{ i} - {ξ}^{ i} ) ] + \\ \{ {[ {K}_{H} ( {η}_{H}^{ i} - {ξ}^{ i} ) + {K}_{L} ( 1 - {η}_{H}^{ i} - {ξ}^{ i} ) ]}^{2} - \\ {4 {K}_{H} {K}_{L} {ξ}^{ i} ( 1 - {ξ}^{ i} ) \}}^{\frac{1}{2}}\end{matrix} \}$$

If left-hand-side is then normalized to K_{H}ηⁱ_{H}+K_{L}(1−ηⁱ_{H}), the effective affinity at infinite dilution, both x- and y- intercepts converge to 1. However, the bulk of the curves differ from each other unless K_{H} and K_{L} are equal, and their curvature and transition depend on the relative magnitude of K_{H} and K_{L} at varying η.

1-2-3. Application of the Theoretical Description: Two-State Model

The theoretical model permits the analysis of adsorption isotherms in terms of any number of populations of receptors. However, the two-state model captures the salient features of the data described herein, thereby providing a firm theoretical basis for the analysis in terms of the straight-line asymptotes of the Scatchard plots constructed from the mAb-I and mAb-II reference assays described in connection with FIGS. 7 and 8. This is demonstrated by performing a regression analysis of the same data in terms of the two-state model, yielding the fits shown as solid lines in those figures.

The theoretical model thus provides a useful parameterization of receptor heterogeneity in terms of affinities and values of receptor coverage. As properties of the carrier population change over time, such changes can be tracked by repeatedly performing a reference assay as a quality control measure providing updated parameters.

Crossover Locus

The transition (or “crossover”) separating high-affinity and low-affinity regimes in each of the subpopulations of increasingly higher coverage, apparent in FIGS. 10A and 10B, may be further characterized in terms of the locus of crossover points. Defining, for the subpopulation of i-th coverage, the cross-over point as the point of intersection of the asymptotes to the respective high-affinity and low-affinity branches in the Scatchard plot, the locus of crossover points as a function of varying i is also related to the heterogeneity characteristics of the entire population.

In the following discussion, the superscript i is dropped for convenience for variables, R_{H}ⁱ, R_{L}ⁱ, Rⁱ, and for the auxiliary variables

$$z ≡ \frac{{C}_{lr}^{i}}{{C}_{l}} and B ≡ {C}_{lr}^{ i} .$$

In terms of variables, z and B, expressions for the asymptotes have the form:
z=z_{H}−\tilde{K}_{H}B and z=−\tilde{K}_{L}(B-B_{L}),
where

$$\begin{matrix}{z}_{H} = {R}_{H} {K}_{H} + {R}_{L} {K}_{L} , & {\tilde{K}}_{H} = \frac{{R}_{H} {K}_{H}^{2} + {R}_{L} {K}_{L}^{2}}{{R}_{H} {K}_{H} + {R}_{L} {K}_{L}} , \\ {B}_{L} = R = {R}_{H} + {R}_{L} , & {\tilde{K}}_{L} = \frac{{K}_{H} {K}_{L} R}{{R}_{H} {K}_{L} + {R}_{L} {K}_{H}} ,\end{matrix}$$

The solution of the interception point, (B_{c}, z_{c}), as an function of ηⁱ and Cₗᵣₒⁱ, is:

$$\begin{matrix}{B}_{c} = \frac{{z}_{H} - {\tilde{K}}_{L} {B}_{L}}{{\tilde{K}}_{H} - {\tilde{K}}_{L}} \\ {z}_{c} = \frac{{\tilde{K}}_{L} ( {\tilde{K}}_{H} {B}_{L} - {z}_{H} )}{{\tilde{K}}_{H} - {\tilde{K}}_{L}}\end{matrix}$$

The crossover locus, constructed from these expression using the values of K_{H}, K_{L}, R_{H}ⁱ and R_{L}ⁱ produced by regression analysis in terms of the two-state model (FIG. 10A), is shown in FIG. 10B. Specifically, in the limit R →0, the locus is seen to intercept the origin, implying that the existence of (at least) two states persists even in the regime of very low coverage, and therefore suggesting that the nature of these two states is associated with individual receptor molecules, such as orientation or epitope accessibility, rather than with a “collective” effect, for example the selection of receptor configurations of increasingly smaller footprint with increasing receptor coverage (“crowding”): in that situation, as coverage decreases, steric constraints would be expected to diminish and disappear as coverage decreases, placing the intercept of the crossover locus at a “characteristic” finite value of coverage. That is, the low coverage behavior of the crossover locus points to the nature of the underlying receptor heterogeneity.

Shape of Distribution

Adsorption isotherms of carrier subpopulations derived from a reference assay such as those described in connection with FIG. 10A, contain information related to changes in shape of the intensity distribution with varying ligand concentration.

In the Langmuir regime of the adsorption isotherm, i.e., when the decrease in the initial solution concentration of ligand due to receptor binding is negligible, one has Cₗ₀≦≦Cᵣ₀, and Cₗ≡Cₗ₀ⁱ. Then, {ξⁱCᵣ₀ⁱ} denotes the intensity distribution, or the “intensity histogram”, at any given ligand concentration, C₁₀. Only if ηⁱ, the ratio of high affinity to low-affinity receptors, is identical for all values of receptor coverage, will ξⁱ not depend on Cᵣₒⁱ (or i), and will {ξⁱCᵣ₀ⁱ}, the intensity histogram, display similar shapes at different ligand concentrations. However, as now shown, if ηⁱ is Cᵣ₀ⁱ (or i)-dependent, the intensity histogram will change shape as ligand concentration increases.

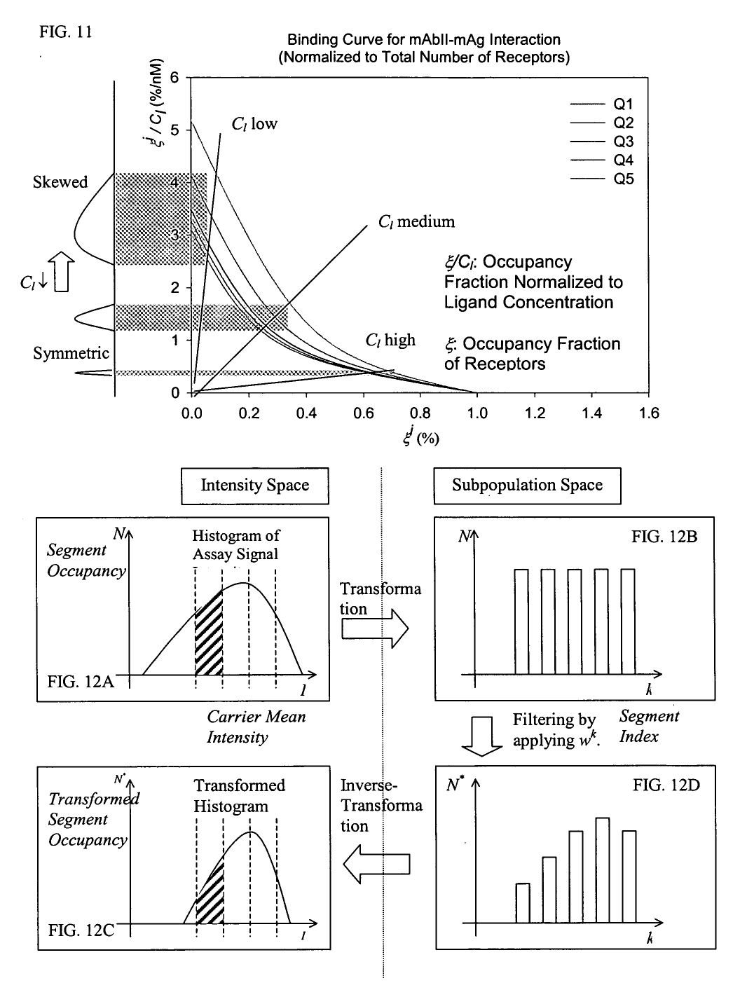

To determine how the shape of the distribution evolves, one invokes the expressions given above for the occupancy fraction and the crossover locus in the two-state model. FIG. 11, representing the set of fits for the data set in FIG. 10B, normalized to R, shows that, in the limit of vanishing ligand concentration, illustrated in the figure by a steep straight line marked “Cₗ low”, the y-intercepts of the fits to the five subpopulations vary over a wide range in such a manner that ξ^{k}'s are spaced more widely apart as k increases. As illustrated in the figure, the effect of this dispersion in the occupancy fractions is a corresponding skew in the intensity distribution. On the other hand, at high ligand concentration, illustrated in the figure by a straight line marked “Cₗ high”, the y-intercepts of the fits to the five subpopulations vary over only a limited range and display a smaller dispersion, producing a more symmetrical shape of the intensity distribution. This ligand concentration-dependent dispersion of occupancy fractions on the change in peak shape agrees with the experimental results shown in FIGS. 6 (A)-(D).

The function η(R) (“η-plot”), representing the heterogeneity characteristics intrinsic to the parent carrier reservoir, relates to receptor coverage only, that is, it neither depends on affinity constants nor on ligand concentration. An evolution of the shape of the intensity distribution with varying ligand concentration in accordance with the predictions of FIG. 11 represent an indicator of such heterogeneity in the receptor population.

Choice of S: Carrier Redundancy vs. Segmentation Frequency

To reliably characterize the heterogeneity in a given receptor population by application of the segmentation method of the invention, it generally will be preferable to employ a large number of carriers. According to the statistical theory of sampling distributions, the error in the estimates of the sample mean (and other moments) and the associated confidence interval(s), are related to sample size. The minimal number of carriers required should be sufficiently large so as to ensure that a specified confidence interval placed upon the estimate of the mean contains the actual population mean. As S increases, the number of carriers per subpopulation decreases, and the uncertainty associated with computed subpopulation mean values correspondingly increases: when standard deviations of those estimates exceed the difference in mean values of adjacent segments, the segmentation process is no longer a well defined one.

Related to this condition is the requirement that η-plots of adjacent segments must not intersect, in order to ensure the same ordering of Rₖ and segment indices, k, and ensuring a one-to-one correspondence between segment indices for different histograms, as discussed above. The effective affinities, \tilde{K}_{L} and \tilde{K}_{L}, computed using the parameters produced by regression analysis of the data in FIG. 10A in terms of the two-state model, are shown in FIG. 10C as a function of receptor coverage, confirming that asymptotic slopes are nearly constant.

Thus, given an initial choice of carriers of a specific type, the value S of segments is thus limited by a certain maximal value, Sₘₐₓ, corresponding to a minimal number of carriers (of given type) in each segment, permitting the evaluation of carrier subpopulation mean values to be within a desirable statistical confidence. In practice, it will be desirable to have a greater total number of carriers than the minimum discussed above. Preferably, in the READ embodiment of the invention, each subpopulation will contain at least 20 carriers of each type, more preferably at least 50 carriers of each type.

The resolution attained by η^{k}(R^{k}) as an approximation to the underlying continuous function, η(R), illustrated in FIG. 10E, is thus limited by the condition that each of the subpopulations contain a certain minimal number of carriers. On the other hand, increasing S beyond the value ensuring that the highest frequency component of the band-limited continuous function η(R) is accurately sampled will have no effect on further improving resolution. In fact, the critical sampling frequency defines an upper bound for Sₘₐₓ. FIG. 10F compares the two discrete functions of η^{k}(R^{k}) obtained for the test assay involving mAb-II using S=5 and S=10, with substantially identical shape.

2. Filtering Sample Intensity Histograms

The method of filtering the signal intensity histogram produced in a sample assay by application of the steps is summarized in FIG. 12. Following segmentation of the histogram into equal subpopulations, weight function, {w^{k}; 1≦k≦S} is constructed by selection of a mapping, η^{k}→w^{k}. The weight function provides a prescription for the relative suppression of contributions to the assay signal distributions from (subpopulations of) carriers displaying a large fraction of low affinity receptors (and hence the relative enhancement of contributions from (subpopulations of) carriers displaying a large fraction of high affinity receptors). Filtering, performed by application of the weight function, modifies the subpopulation occupancies in order to enhance discrimination by assigning different weights to signals contributed by subpopulations of carriers of varying total receptor coverage, identified and parameterized in accordance with the methods disclosed in the previous section. While the theoretical model permits analysis and filtering operations to be based on two or more states, one proceeds here with the two-state model which was shown in connection with the analysis of the data in FIGS. 10-11 to capture the salient aspects of the heterogeneity in receptor populations.

Underlying this filtering method is the assumption that all samples of receptor-functionalized carriers taken from the same reservoir (“parent population”) of carriers display the same degree of heterogeneity. Accordingly, the sample assay histogram is assumed to reflect the degree of heterogeneity previously characterized in the reference assay and parameterized in terms of the two-state (or generally n-state) model of the present invention. That is, the same mapping and weight functions apply to the transformation of any distribution of signal intensities produced by subsets of carriers extracted from such a parent population. A generalization of this method applying to the detection of heterogeneity in the ligand population is described below.

2-1 Preprocessing

Reference and sample assays are performed, not necessarily at the same time, and not necessarily in that order. In the preferred READ format, images are recorded from the array showing the pattern of ligand binding to carrier-immobilized receptors as a function of ligand concentration. Preprocessing steps are applied to the images as necessary using conventional methods to remove spatial variations in intensity reflecting, for example, non-uniformities in the illumination intensity profile (“flat-fielding”) and fluctuations reflecting “cross-talk” between adjacent beads and geometric effects reflecting, for example, the spherical shape of beads. Intensities recorded are then sorted in ascending order to form a 1-D array.

An optional anomaly (outlier)-removal step is then applied to this array. First, intensity differences are calculated for each pair of adjacent elements in the presorted intensity array to form an array of differences. The average difference is obtained and a threshold value set by multiplying the average difference by an adjustable factor, say 5. Next, in the array of differences, starting from elements whose positions correspond to preset minimal and maximal percentiles, say 20% and 80%, of the intensity array, one searches for the first element that exceeds the threshold. If found, the position of the element is marked as a new boundary. If not found, the end of array is the new boundary. In the intensity array, all elements falling outside the new boundaries are rejected as outliers.

2-2 Filtering

2-2-1 Histogram Segmentation

To transform the sample assay signal, one first segments the preprocessed intensity distribution into S subpopulations of equal area (FIG. 12, upper left panel) in the same way as described above for the reference assay distribution. The number S is chosen so as to ensure that the number of carriers in each segment exceeds a certain preset minimum in order to retain statistically meaningful carrier subpopulations per segment.

This segmentation step corresponds to a transformation of the assay intensity distribution from an intensity representation (I_{b}, N_{b}), the index b counting histogram bins, into a subpopulation representation, (k, Nₖ), in which the abscissa represents segment indices and the ordinate represents the area (or subtotal of carriers) of each segment. By construction, segment areas are initially identical, that is, Nₗ=. . . =Nₖ=. . . =Nₛ. Each of the S subpopulations in the segmented sample histogram is matched to a corresponding subpopulation in the segmented reference histogram.

2-2-2 Filter Construction: Weight Function

Central to the filtering operation is a weight function that is associated with the heterogeneity of the parent population characterized with the reference assay. Defined herein is a weight function as a set of S coefficients, {w^{k}, 1≦k≦S}, to be multiplied to the subpopulation occupancies in order to emphasize contributions from the “high affinity” portion of the receptor population. The weight function w(k) is derived from w(R^{k}), which represents a “stretched” version of function η^{k}=η(R^{k}). That is, as with histogram “stretching”, familiar in the context of image analysis, the η axis is “stretched” in order to extend the range of the function η(R), generally by employing a monotonic mapping that assigns a value of w^{k} to each segment index, k.

Many functional forms of the mapping η→w may be considered, in a manner analogous to pixel histogram transformation “stretching” familiar in the context of image analysis (Seul, O'Gorman & Sammon, “Practical Algorithms for Image Analysis”, Cambridge University Press, 2000). Examples include:

1. A step function placing full weight to segments, k≧k_{Threshold}, and assigning a weight of zero to other segments;

2. A monotonically increasing function, such as a straight line with an offset, or a polynomial function.

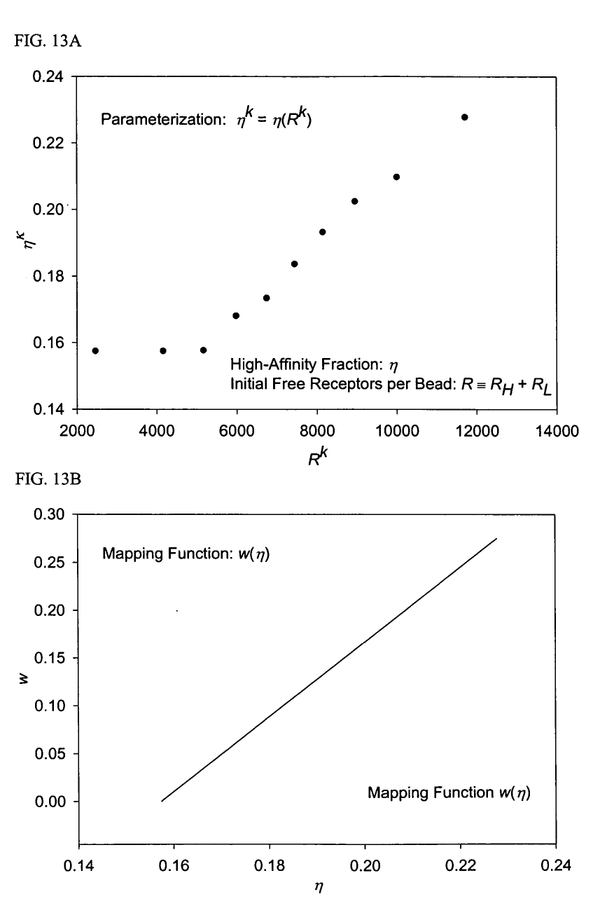

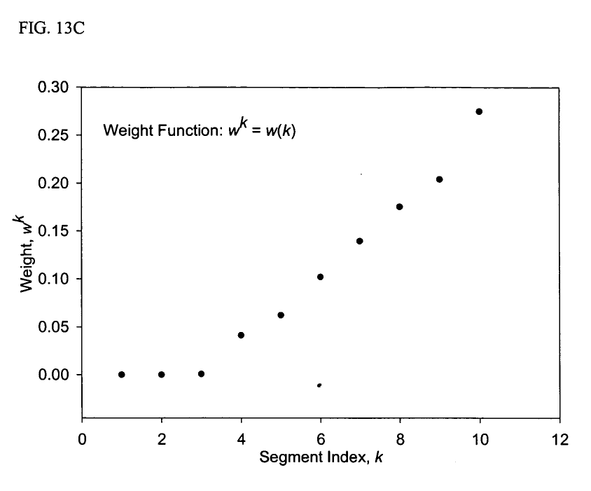

FIG. 13 illustrates an example of the construction of a weight function w^{k}=w(R^{k}) (FIG. 13C) from the parameterization η^{k}=η(R^{k}) (FIG. 13A) by application of a linear mapping function w=w(η) (FIG. 13B) for the data recorded in a reference assay of mAb-II binding to beads displaying HLA Class-II antigens (FIG. 6).

2-3 Transformation and Calculation of Moments

The next step is to apply the weight coefficients to the sample assay signal intensity distribution; the transformed segment occupancy is thus the product of the area of the segment and its corresponding weight. A set of moments of the transformed histograms can be computed using the expressions described below.

Assuming the mean intensity, carrier count, and variance of those S subpopulations are (\overline{x}₁,n₁,σ₁), (\overline{x}₂,n₂,σ₂) . . . (\overline{x}ₛ,nₛ,σₛ), the new moments, (\overline{x}, n, σ), associated with the above transformed subpopulations, each being assigned a weight {w^{k}1≦k≦S}, are:

$$\begin{matrix}n = {\sum }_{k = 1}^{S} {w}_{k} {n}_{k} = 〈 {n}_{k} 〉 , \\ \overline{x} = {\sum }_{k = 1}^{S} {\sum }_{{i}_{k} = 1}^{{n}_{k}} {w}_{k} {x}_{k , {i}_{k}} = {\sum }_{k = 1}^{S} {w}_{k} \overline{{x}_{k}} = 〈 \overline{{x}_{k}} 〉 , and \\ {σ}^{2} = {\sum }_{k = 1}^{S} {\sum }_{{i}_{k} = 1}^{{n}_{k}} {{w}_{k} ( {x}_{k , {i}_{k}} - \overline{x} )}^{2} = {\sum }_{k = 1}^{S} {w}_{k} {\sum }_{{i}_{k} = 1}^{{n}_{k}} {\{ {x}_{k , {i}_{k}} - [ {x}_{k} + ( \overline{x} - \overline{{x}_{k}} ) ] \}}^{2} \\ = {\sum }_{k = 1}^{S} {w}_{k} [ {σ}_{k}^{2} + {( \overline{x} - \overline{{x}_{k}} )}^{2} ] \\ = {\sum }_{k = 1}^{S} {w}_{k} {σ}_{k}^{2} + {\sum }_{k = 1}^{S} {{w}_{k} ( \overline{x} - \overline{{x}_{k}} )}^{2} \\ = 〈 {σ}_{k}^{2} 〉 + 〈 {( \overline{x} - \overline{{x}_{k}} )}^{2} 〉\end{matrix}$$

Following the completion of this step, the filtered subpopulation representation may be transformed back into the conventional representation showing a histogram of S bins (FIG. 12). The k-th segment in the filtered histogram has a height (frequency density) of \overline{C}ₗᵣ^{(k)}=w₍ₖ₎C^{(k)}ₗᵣ and a range and width specified by min(C^{(k)}ₗᵣ) and max(C^{(k)}ₗᵣ).

2-4 Implementation

2-4-1 Flow Chart

A flow chart of the sequence of processing steps in this heterogeneity-filtering algorithm (“HetFilter”) is shown below.

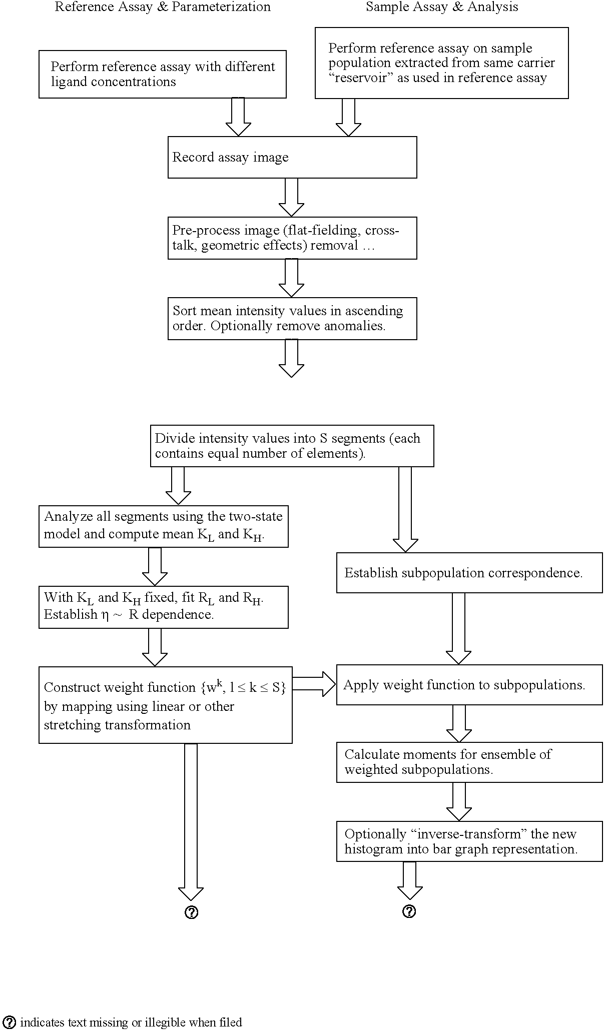

[symbol] [symbol] 2-4-2 Pseudocode Het Descriptor

main( )
{

ReferenceAssayParametrization(optS, RefAssayImage)
SampleAssayAnalysis(optS, SampleAssayImage)

}
ReferenceAssayParametrization(S, AssayImage)
{

| /* Image preprocessing */ | |
|---|---|
| ApplyFlatFieldCorrection(AssayImage); | /* Remove image non-uniformities */ |
| ApplyCrossTalkCorrection(AssayImage); | /* Remove cross-talk between carriers */ |
| ApplyGeometryCorrections(AssayImage); | /* Remove geometric effects, such as |
| | “edge effect”; etc */. |
| ExtractIntensities(AssayImage, tmpIntArray); | |
| SortIntensity(tmpIntArray, IntArray); | |
| optOutlierRemoval(IntArray, sIntArray) | /* Optional Step */ |

ComputeHistogram(sIntArray, Histogram);
/* Calculate the high-affinity fraction dependence on coverage */
FOR (number of divisions, S)
{

| /* Segment preprocessed histogram; */ | | |
|---|---|---|
| SegmentHistogram(S, Histogram, sHistogram); | | |
| Calculate Cₗᵣ/Cₗ; | | |
| Fit Cₗᵣ/Cₗ vs Cₗᵣ to the two-state model; | | |
| Obtain K_{L}, K_{H}; | | |
| /* Confirm among different segments, K_{H} and K_{L} are similar */ | | |
| Const MaxDevK; | /* | “ChiSquared” |

Threshold */

If((EvalDevK(ArrayOfK_{L}, S)>MaxDevK) OR (EvalDevK(ArrayOfK_{H}, S)>
MaxDevK))
{

ReferenceAssayParamerizationHetLigand(optS, RefAssayImage);
Return w, η, S, MapFunction;
Exit Subroutine;

}
/*Calculate average K_{H} and K_{L} */
K_{L} = Average(ArrayOfK_{L}, S);
K_{H} = Average(ArrayOfK_{H}, S);
/* Keeping K_{L} and K_{H} fixed */
FOR (each of S segments, indexed by k)
{

/* Fit Cₗᵣ/Cₗ vs Cₗᵣ */
/* Evaluate high-affinity fraction, η(k) = R_{H}(k)/R(k) */
η = TwoStateModel(Cₗᵣ/Cₗ, Cₗᵣ, FitParameters);

}
/* Determine uncertainty in determining η^{k}(R^{k}) to stop at optimal S */
If (number of beads per segment < certain minimum) Break Loop;
If (fitted Scatchard curves among neighboring segments starts to cross)

Break Loop;

S ++;

| } | |
|---|---|
| ConstructLookupTable(η, R, S); | /* For optimal S, |
| | establish lockup table of |
| | (R^{k}, η^{k}) */ |
| Construct WeightFunction(w, η, S, MapFunction); | /* Construct weight, Wₖ, |
| | as a function of k, where |
| | k=1, . . . , S, using mapping |
| | method */ |
| Return w, η, S, MapFunction; | |
| /* End Subroutine */ | |

}
optOutlierRemoval(IntArray, sIntArray)
{

| /* Preprocess histogram (optional) by removing outliers outside of a “protected | |
|---|---|
| zone” delineated by preset “loZoneIndex” and “upZoneIndex” */ | |
| /* Set threshold by multiplication of average difference by a factor, say 5 */ | |
| Threshold = CalculateDifferenceArray(IntArray, ScaleFactor, DiffArray); | |
| index = loZoneIndex; | |
| WHILE( (index>loLimit) AND (DiffArray(index) < Threshold) ) index−−; | |
| loZoneIndex = index; | /* Mark lower limit of “safe zone” */ |
| WHILE( (index<upLimit) AND (DiffArray(index) < Threshold) ) index++, | |
| hiZoneIndex = index; | /* Mark upper limit of “safe zone” */ |

/* Crop data within new boundaries */
CropArray (loZoneIndex, hiZoneIndex, IntArray, sIntArray);

}
ReferenceAssayParamerizationHetLigand(optS, RefAssayImage)
{

/* Compute the heterogeneity in ligand population that gives rise to the
inhomogeneous K_{H} and K_{L}'s in different segments */

}
SampleAssayAnalysis(AssayImage, optS)
{

| /* Image preprocessing */ | |
|---|---|
| ApplyFlatFieldCorrection(AssayImage); | /* Remove image non-uniformities */ |
| ApplyCrossTalkCorrection(AssayImage); | /* Remove cross-talk between carriers */ |
| ApplyGeometryCorrections(AssayImage); | /* Remove geometric effects, such as |
| | “edge effect”; etc */. |
| ExtractIntensities(AssayImage, tmpIntArray); | |
| SortIntensity(tmpIntArray, IntArray); | |
| optOutlierRemoval(IntArray, sIntArray) | /* Optional Step */ |

ComputeHistogram(sIntArray, Histogram);
/* Segment preprocessed histogram; */
/* S denotes number of segments, e.g. 5, 10, 20, 50 */
SegmentHistogram(S, Histogram, sHistogram);
FOR (k=0; k<S, k++)
{

| | |
|---|---|
| TransformHistogram(N, sHistogram, S); | /* (N, k) representation */ |
| FilterHistogram (N, w, S); | /* Apply weight function to |

modify N */

| | |
|---|---|
| CalculateMoments (N, S); | /* Calculate moments (mean |
| | and standard deviation for |
| | “filtered” histogram */ |
| rTransformHistogram(N, sHistogram, S); | /* Back-transform histogram */ |

}

}

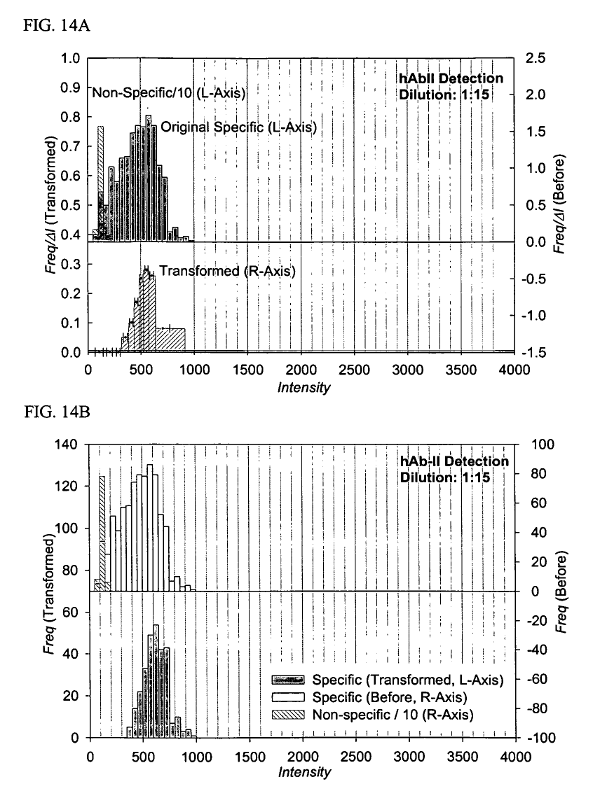

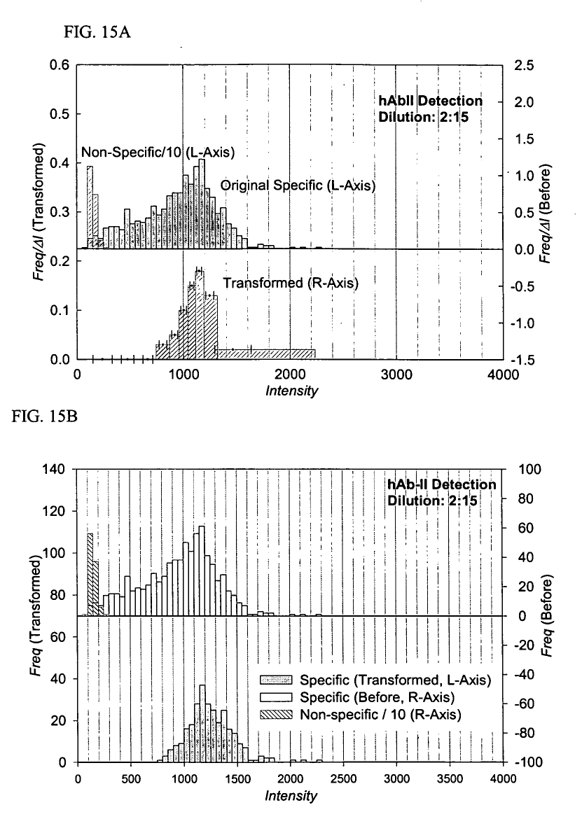

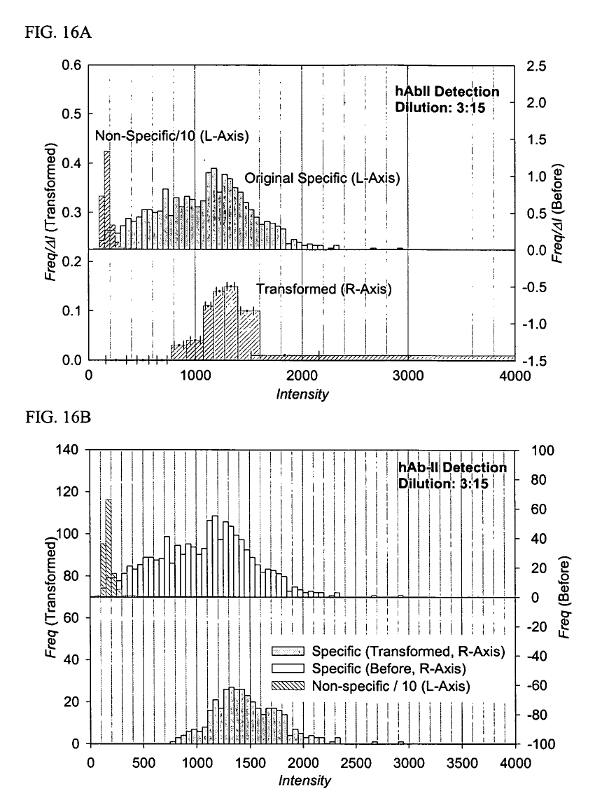

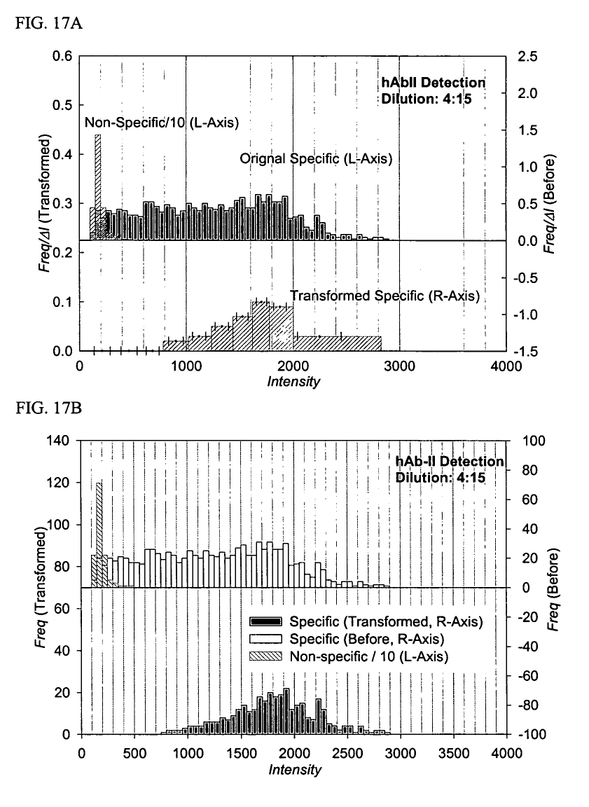

FIGS. 14-17 illustrate the effect of the filtering operation on the hAb-II data recorded using four different dilution factors (FIG. 2). Two forms of the transformed histogram are shown, namely, in the respective upper panels, an “intermediate” representation using S=10 bins, and, in the respective lower panels, a “final” representation using a large number of bins permitting direct comparison to the original histogram.

For all dilution ratios, filtering substantially reduces the width of the original histogram, thereby revealing the “true” intensity and produces corrected estimates of the mean. Values for the mean and variance calculated before and after filtering (Table I) reveal that the transformation greatly improves discrimination without displaying significant sensitivity to the number, S, of segments employed in the parameterization.

TABLE I
Effect of Filtering on the Statistics of Original and
Transformed Intensity Histograms

| | | | | New | New | |
|---|---|---|---|---|---|---|
| Dilution | | Raw | 3σ- | Method | Method | |
| Ratio | Moments | Data | rejection | S = 5 | S = 10 | “Actual” |
| 1:15 | Mean | 417.32 | 428.96 | 579.96 | 569.72 | ˜570 |
| | σ | 177.42 | 139.83 | 110.55 | 110.19 | ˜110 |
| | CV | 43% | 33% | 19% | 19% | ˜19% |
| 2:15 | Mean | 904.32 | 948.13 | 1218.77 | 1201.26 | ˜1200 |
| | σ | 356.40 | 262.82 | 216.82 | 214.23 | ˜214 |
| | CV | 39% | 28% | 18% | 18% | ˜18% |
| 3:15 | Mean | 1030.04 | 1038.28 | 1415.99 | 1424.85 | ˜1425 |
| | σ | 463.07 | 349.11 | 346.13 | 345.47 | ˜345 |
| | CV | 45% | 34% | 24% | 24% | ˜24% |
| 4:15 | Mean | 1206.79 | 1203.97 | 1793.29 | 1754.56 | ˜1750 |
| | σ | 622.76 | 509.39 | 405.67 | 406.26 | ˜407 |
| | CV | 52% | 42% | 23% | 23% | ˜23% |

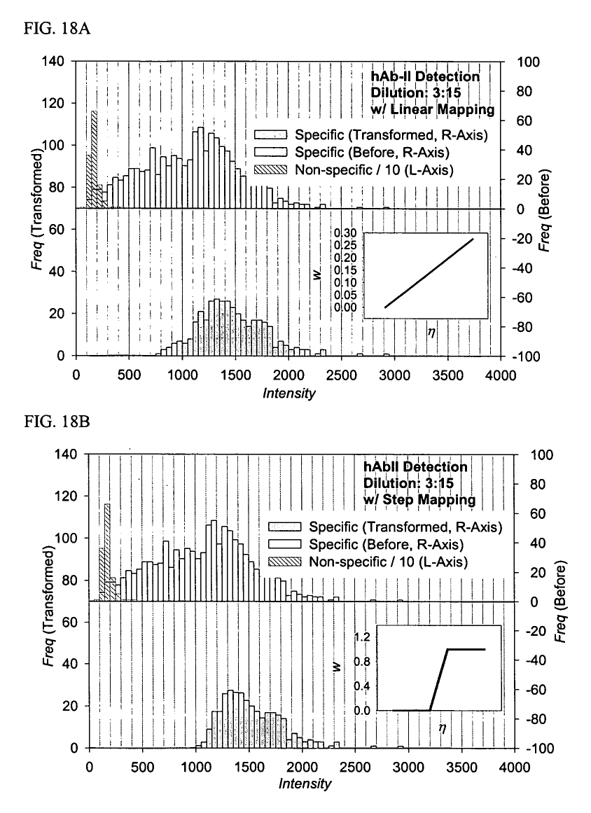

The effect of the shape of the mapping η→w on the shape of the filtered histogram is illustrated in FIG. 18 for the case of the hAbII data recorded for a dilution of 3:15, first shown in FIG. 2. In contrast to the linear mapping (inset, FIG. 18A), the non-linear mapping (inset, FIG. 18B) places greater weights on contributions from (subpopulations of) carriers having a larger fraction of high-affinity receptors while attenuating contributions from (subpopulations of) carriers below the 50th percentile of the population.

2-5 General Case: Heterogeneity in Ligand Population

In the version of the filtering method described so far, one has assumed that heterogeneity observed in the signal intensity distributions produced in receptor-ligand interaction assays is dominated by heterogeneity in the receptor population. In accordance with this embodiment of the method, sample assay intensity histograms are filtered simply by applying the weight function derived from the parameterization of the corresponding reference assay.

More generally, as described below, the disclosed method also addresses the case in which non-uniformities in the ligand population contribute in significant measure to the overall heterogeneity, manifesting itself in the form of broad and skewed intensity histograms and, in the READ format, in spatial non-uniformities visible in assay images. The effect of heterogeneity in the ligand population on the signal intensity distribution may be determined in accordance with the methods disclosed herein by realizing that heterogeneity in the ligand population will be independent from the heterogeneity of any particular carrier-displayed receptor population. The regression analysis described in Sect. I may be repeated with a larger number of parameters to allow for the existence of more than a single constituent in the ligand population. That is, this method permits detection of heterogeneity in the ligand population. Further, the shape of the intensity distribution reflecting ligand heterogeneity may be estimated once the multiple relevant affinities have been estimated, as in the example of the 2×2 matrix of affinities discussed in greater detail below.

2-5-1 Extension of Theoretical Description to General Case

Assuming the presence of two levels of heterogeneity, namely receptor coverage, indexed by i, and affinity constant, indexed by j, the theoretical description in Sect. 1-2-2 presented expressions for affinity constants in terms of mass action:

$$\frac{{C}_{lr}^{i , j}}{( {C}_{r 0}^{i , j} - {C}_{lr}^{i , j} ) {C}_{l}} = {K}^{j} .$$

yielding, for each carrier of i-th coverage, the number of receptor-ligand complexes in the form:

$$\frac{{C}_{lr}^{i , j}}{{C}_{l}} = \frac{{K}^{j} {C}_{r 0}^{i , j}}{1 + {K}^{j} {C}_{l}} and \frac{{C}_{lr}^{i}}{{C}_{l}} = {\sum }_{j} \frac{{C}_{lr}^{i , j}}{{C}_{l}} = {C}_{ro}^{i} {\sum }_{j} \frac{{K}^{j} {η}^{i , j}}{1 + {K}^{j} {C}_{l}}$$

The general case considers the effect of heterogeneity in the ligand population, associated with different affinity constants reflecting, for example, the presence of a mixture of ligands (as in a serum containing polyclonal antibodies with different prevalent epitopes or the partial deterioration of some ligands. It is important to note that the heterogeneity in the ligand population is independent of that in the receptor population. Indexing the different subpopulations by q, the law of mass action produces a modified expression for the affinity constants in terms of mass action:

$$\frac{{C}_{lr}^{i , j , q}}{( {C}_{r 0}^{i , j} - {C}_{lr}^{i , j , q} ) {C}_{l}^{q}} = {K}^{j , q}$$

Before proceeding to compute the ratios, defined are a separate mixing coefficient η_{q}, indexed by q:

$${η}_{q} ≡ \frac{{C}_{l}^{q}}{{C}_{l}}$$

Here, a subscript is used to distinguish it from η^{ij}.η_{q} does not involve indices, i and j; note, that because of the independence of the heterogeneity in the receptor population and that in the ligand population:

$${\sum }_{i , j , q} {η}^{i , j} {η}_{q} = {\sum }_{i , j} {η}^{i , j} \cdot {\sum }_{q} {η}_{q} = 1$$

Hence, the above equation becomes:

$$\frac{{C}_{lr}^{i , j , q}}{( {C}_{r 0}^{i , j} - {C}_{lr}^{i , j , q} ) {C}_{l}} = {η}_{q} {K}^{j , q}$$

$$Then , \\ \frac{{C}_{lr}^{i , j , q}}{{C}_{l}} = \frac{{η}_{q} {K}^{j , q} {C}_{r 0}^{i , j}}{1 + {K}^{j , q} {C}_{l}}$$

$$Thus , \\ \frac{{C}_{lr}^{i}}{{C}_{l}} = {\sum }_{j , q} \frac{{C}_{lr}^{i , j , q}}{{C}_{l}} = {C}_{r 0}^{i} {\sum }_{j , q} \frac{{η}^{i , j} {η}_{q} {K}^{j , q}}{1 + {K}^{j , q} {C}_{l}}$$

The analogy of the special case of the 2-state-model described for receptor heterogeneity involving high-affinity and low-affinity states in the receptor population now involves “high-efficiency” and “low-efficiency” states in the-ligand population. For this 2×2 state, one can express the ratio of total number of occupied sites (or the histogram bin height) and ligand concentration, for the i-th subpopulation, in the form:

$$\frac{{C}_{lr}^{i}}{{C}_{l}} = {C}_{r 0}^{i} [ \begin{matrix}\frac{{η}^{H} {η}_{H} {K}^{HH}}{1 + {K}^{HH} {C}_{l}} + \frac{{η}^{H} ( 1 - {η}_{H} ) {K}^{HL}}{1 + {K}^{HL} {C}_{l}} + \\ \frac{( 1 - {η}^{H} ) {η}_{H} {K}^{LH}}{1 + {K}^{LH} {C}_{l}} + \frac{( 1 - {η}^{H} ) ( 1 - {η}_{H} ) {K}^{LL}}{1 + {K}^{LL} {C}_{l}}\end{matrix} ]$$

where η^{H} is the high-affinity fraction in terms of receptor coverage as in two-state (receptor) model. η_{H} is the fraction of “high-efficiency” ligand. Affinity constants thus correspond to the following interactions suggested by the binding and cross-binding activities among four identities, where R represents receptors and L represents ligands. The above equation, mathematically, is equivalent to a reaction between a homogeneous ligand population and a heterogeneous four-receptor population.

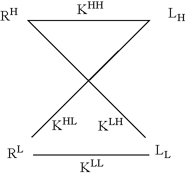

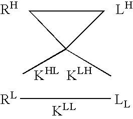

The set of affinity constants corresponding to this configuration is equivalent to the co-affinity matrix disclosed in “Multianalyte Molecular Analysis Using Application-Specific Random Particle Arrays,” U.S. Application Ser. No. 10/204,799, filed on Aug. 23, 2002; WO 01/98765, although the present invention relates to the presence of multiple types (or states) of receptors on a single type of carrier. The special case of a two-state model for receptors and ligands produces a 2×2 matrix,

$$K = [ \begin{matrix}{K}^{HH} & {K}^{LH} \\ {K}^{HL} & {K}^{LL}\end{matrix} ] ,$$

In the special case of a homogeneous receptor population, then K^{LH}=K^{HH} and K^{LL}=K^{HL}. By combining terms with equal K's and dropping the subscripts, the above equation reduces to

$$\frac{{C}_{lr}^{i}}{{C}_{l}} = {C}_{r 0}^{i} [ \frac{{η}_{H} {K}^{H}}{1 + {K}^{H} {C}_{l}} + \frac{( 1 - {η}_{H} ) {K}^{L}}{1 + {K}^{L} {C}_{l}} ] .$$

This expression has the same form as that which describes the reference experiment in the two-state (receptor) model. One can thus characterize the heterogeneity in the ligand population in terms of a mixing coefficient, η_{H}, which may be determined in a reference experiment using an “ideal” test receptor to probe the heterogeneity in the ligand population.

Consider a more general case in which the heterogeneity in ligand is assumed to be continuous and is characterized by a density function θ(q), one has:

$$\begin{matrix}\frac{{C}_{lr}^{i}}{{C}_{l}} = {C}_{r 0}^{i} {\sum }_{j} \int ⅆ {η}_{q} \frac{{η}^{i , j} {K}^{j} ( q )}{1 + {K}^{j} ( q ) {C}_{l}} \\ = {C}_{r 0}^{i} \int θ ( q ) ⅆ q {\sum }_{j} \frac{{η}^{i , j} {K}^{j} ( q )}{1 + {K}^{j} ( q ) {C}_{l}}\end{matrix}$$

Note that θ(q) is independent of index i and can be factored out of the summation. In the special case of two distinct receptor types (or states) displayed on carriers, the above equation is thus reduced to

$$\frac{{C}_{lr}^{i}}{{C}_{l}} = \int θ ( q ) ⅆ q \cdot {C}_{r 0}^{i} [ \frac{{η}^{i , H} {K}^{H} ( q )}{1 + {K}^{H} ( q ) {C}_{l}} + \frac{( 1 - {η}^{i , L} ) {K}^{L} ( q )}{1 + {K}^{L} ( q ) {C}_{l}} ]$$

For any given q, if there is η^{i,H} is independent or monotonically increasing as a function of Cᵣₒⁱ, or even though η^{i,H} decreases as Cᵣₒⁱ increases but the product, (η^{i,H} Cᵣₒⁱ) is constant or monotonically increasing, there should exist the same ranking of the quantity, ƒ(i,q),

$$f ( i , q ) = {C}_{ro}^{i} [ \frac{{η}^{i , H} {K}^{H} ( q )}{1 + {K}^{H} ( q ) {C}_{l}} + \frac{( 1 - {η}^{i , H} ) {K}^{L} ( q )}{1 + {K}^{L} ( q ) {C}_{l}} ]$$

as that of the value of Cᵣₒⁱ, given K^{H}>K^{L}, that is ƒ(m,q)≦ƒ(n,q), for V m<n. In this case, since θ(q) is not i-dependent and hence the integrand has the same ranking as that of the value of Cᵣ₀ⁱ, the result of the integration should have the same ranking as that of the value of Cᵣ₀ⁱ.

The actual peaks recorded from interaction between human sera and carrier population displaying HLA Class-II antigens, as described in Example 2, are broader than those recorded from assays performed using monoclonal antibodies. Those features suggest an additional contribution to the intensity histogram shape arising from heterogeneity in the ligand population. Considering the fact that, in the reference experiment, ηⁱ shows a monotonically increasing dependence on Rⁱ, one concludes that, in the actual experiment involving hAb-II serum, regardless of the detailed form of heterogeneity present in ligand, as characterized by θ(q), the intensity corresponding to the i-th receptor coverage has the same ordering as that of Cᵣ₀ⁱ or that of the index, i. Thus, regardless of the additional heterogeneity present in the ligand population, application of the filtering method of the present invention in order to reduce the effect of receptor heterogeneity on the assay signal intensity histogram using the weight function constructed from the reference assay represents a well-defined process.

2-6 Extension: Pixel-Intensity Distribution

The method, as described so far, addresses transformations of the distributions of carrier mean intensities, [symbol] I [symbol] _{j,k} where j denotes the j-th carrier type and k denotes the k-th of S carrier subpopulations. In this form, the method applies to several assay formats, notably to READ™ and to flow cytometric analysis of sets of encoded microparticles

In the preferred embodiment, READ, spatial non-uniformities manifesting themselves on the “pixel” scale, that is, on a length scales smaller than the carrier size; for example, using typical configurations described in the Examples included herein, the image of a 3 μm-diameter bead comprises tens of pixels. In the experiments described herein with reference to Examples 1-3, spatial non-uniformities were in some instances optically resolvable. In such instances, it may be desirable to analyze signal intensities directly by forming signal intensity distributions based on averaging not over entire carriers such as beads but averaging over “pixel” elements, to obtain pixel mean intensities, [symbol] Iₚ [symbol] _{j,k}.So long as our assumptions, especially the 4th assumption—namely, that receptor-ligand binding events are uncorrelated—remain valid, the methodology described herein also can be applied to the transformation of pixel intensity distributions to further improve assay fidelity and reliability. Such finer levels of resolution are not available in flow cytometric detection methods.

Examples of application of the pseudocode and examples that illustrate other features of making and using the methods are set forth herein below.

### EXAMPLES 1

Reference Assay and Parameterization: Anti-HLA Monoclonal Antibody Titration

Pools of antigens were extracted from cells with known HLA antigens and affixed to color-encoded beads using methods of passive adsorption known in the art. Approximately 500 μg of the protein were incubated with 1 mg of the color-encoded beads in a coupling buffer containing 3 mM sodium chloride, 2 mM sodium phosphate, pH 3.0, and incubated overnight at 37° C. under constant rotation. Bovine serum albumin (BSA) was used as a negative control protein in the coupling reaction. After protein coupling, the particles were washed in phosphate-buffered saline (PBS; 0.1 M sodium phosphate, 0.15 M sodium chloride, pH 7.2) also containing 0.05% Tween-20 (PBST). For array assembly, all of the functionalized beads of interest were combined into a test tube and subjected to Light-controlled Electrokinetic Assembly of Particles near Surfaces (LEAPS) or methods of direct assembly as described (op.cit.). The array was decoded using fluorescence microscopy to detect the signals from the fluorescent coding dyes associated with beads of different types. Unreacted reactive sites were blocked using 1% BSA in PBST prior to conducing the assay.

To perform the reference assay, mouse monoclonal antibody specific for HLA Class I or Class II (also mAb-I and mAb-II) were serially diluted in PBST with 1% BSA, and 1-10% NP-40. Diluted solutions of known concentration of mAb were placed in contact with arrays of HLA-functionalized beads, and incubated in a small (home-built) humidified and temperature-controlled reaction chamber for 1 hour under modest shaking. Unbound antibodies were removed by intensive washing with PBST. The bound antigen-antibody complexes were detected using Cy5-labeled anti-mouse IgG Fab fragments. Titration data are displayed in FIGS. 4 and 5.

### EXAMPLE 2

Actual (“Sample”) Assay and Application of Filtering: Allo-Antibody Profiling

Arrays of HLA-functionalized, color-coded beads were prepared, and unreacted sites blocked as in Example 1. Instead of mouse monoclonal antibodies, human polyclonal antisera specific for HLA Class I or Class II molecules, serially diluted in PBST with 1% BSA, and 1-10% NP-40, were used to establish titration curves. Increasingly dilute sera were placed in contact with arrays of HLA-functionalized beads, and incubated in a small (home-built) humidified and temperature-controlled reaction chamber for 1 hour under modest shaking. Unbound antibodies were removed by intensive washing with PBST. The bound antigen-antibody complexes were detected using Cy5-labeled goat anti-human fragment. Titration data are displayed in FIG. 2E.

### EXAMPLE 3

Profiling of Anti-Jo1 Antibody

A titration curve was established using arrays of color-encoded beads displaying a set of autoantigens and a human serum sample known to contain anti-Jo-1 antibody (as well as possibly other antibodies). A 6-antigen panel was formed by attaching auto-antigens—Jo-1, SSA-60, Sm, Sm/RNP, CENP, and SSB—to core-shell beads by passive adsorption under conditions of low salt and low pH. Following a blocking step with buffer containing 0.1% BSA (w/v), beads were stored individually at 4° C. until the time of array assembly. To form arrays, beads displaying the 6 antigens were pooled and assembled on silicon chips.

A titration curve was obtained using the serially diluted anti-Jo-1 positive serum at dilution ratios of 1:3, 1:9, 1:27, 1:81, 1:243, 1:729, 1:2187 and 1:6561. Diluted sera were incubated with chips for 30 min at room temperature. After removing weakly bound antibodies, an Alexa-labeled goat anti-human IgG was used to visualize captured anti-Jo-1. After a second washing step, decoding and assay images were collected and the assay signals were then extracted and analyzed.

### EXAMPLE 4

Analysis of Images of “Spotted” Probe Arrays

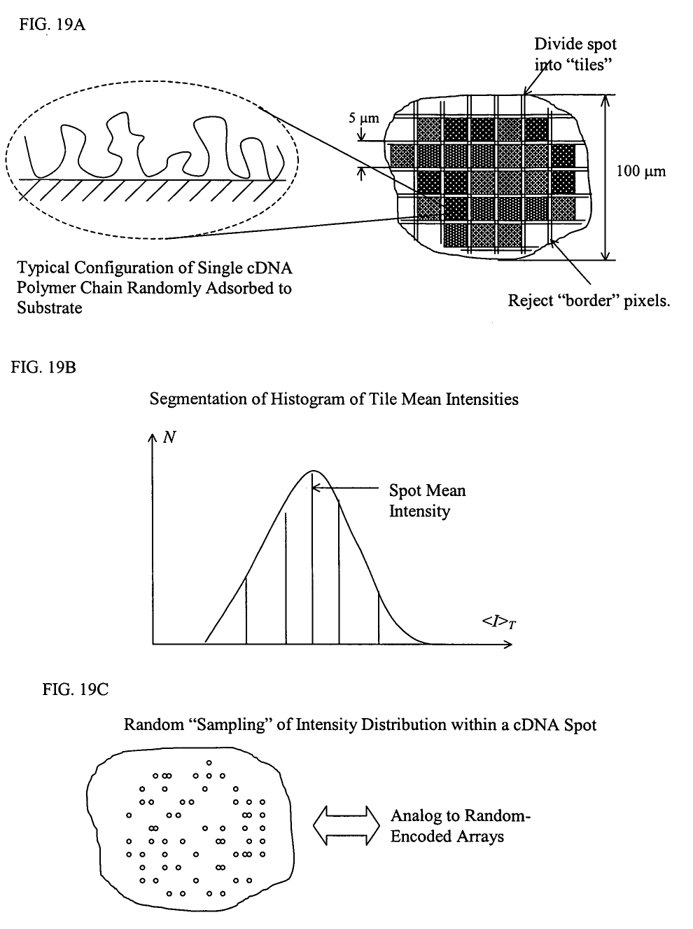

An image of a spotted array produced, for example, by raster scanning the fluorescence intensity distributions of constituent “spots”, the intensity distribution, I=I(x, y), across each spot, as illustrated in FIG. 19A, representing the amount of target captured to the “probe” within that spot, is readily analyzed using the methods of the present invention in order to analyze heterogeneity in the probe population which may manifest itself directly in the form of spatial non-uniformity in the intensity distribution. Spotted (or otherwise produced) spatially encoded probe arrays, composed of long oligonucleotides (such as the 150-nt in length disclosed in U.S. Pat. No. 6,461,812) or composed of cDNA molecules, widely used in the art, contain multiple spots at a typical spot-to-spot separation of, say, 300 μm, each spot of typically, say, 100 μm in diameter containing one species of cDNA. FIG. 19A illustrated such a spotted array, with cDNA attached to the solid surface.

A direct correspondence to the methods of parameterization and filtering method described herein is established by applying a random sampling step to the function I(x, y). For example, overlay the spot image with an opaque mask containing one or more “holes”permitting the sampling of the underlying spot intensity at the aperture position(s), and compute the mean of the sampled portion of I=I(x, y), also referred to as the aperture mean. This sampling operation preferably is performed in software using standard methods. For example, multiple single samples may be taken at randomly selected positions within the spot, optionally ensuring that sampled positions are separated by a pre-selected minimal distance (in order to avoid correlations between spatially proximal samples); alternatively, an entire set of such samples may be taken, and aperture mean values computed, by sampling in a random set of positions within the spot, as illustrated FIG. 19C. Histograms of aperture mean intensities may then be constructed, sorted, segmented, analyzed and filtered in accordance with the methods of the present invention, as illustrated in FIG. 19B and 19C, as with the processing of carrier intensities in the preferred READ embodiment.

It should be understood that the terms, expressions and examples used herein are exemplary only, and not limiting, and that the scope of the invention is defined only in the claims which follow, and includes all equivalents of the subject matter of the claims. Process and method steps in the claims can be carried out in any order, including the order set forth in the claims, unless otherwise specified in the claims.

What is claimed is:

1. A method for determining receptor density (number of receptors, R, per unit area) for receptors having different affinities, where several receptors in a population of receptors are present on the surface of a substrate and said several receptors bind to the same ligand, and where the receptor-ligand interaction of different receptors has different affinities, K, and wherein the population of receptors includes receptors having a low affinity and receptors having a high affinity, comprising:
contacting, in solution, the substrate with the ligands under conditions where ligands bind to the receptors and whereby an optical signal of a particular intensity is generated when a ligand is bound to a receptor;
sampling the signal intensities on the substrate surface using an aperture which admits the optical signal for the sampling;
averaging the signal intensities sampled by the aperture;
dividing, for a particular known ligand concentration in the solution, the averaged signal intensities into a fixed number of segments, wherein each segment includes samplings within a particular range of signal intensities, and repeating this step at several ligand concentrations to generate several sets of segments;
determining the average of the affinity constants for the low affinity and high affinity receptors, respectively designated K_{L} and K_{H}, from the sets of segments;
determining, for a particular set of segments (designated the k-th set), the receptor density of the low affinity receptors, R_{L}^{(k)}, and of the high affinity receptors, R_{H}^{(k)};
determining, R_{H}^{(k)}/R^{(k)} and/or R_{L}^{(k)}/R^{(k)}, i.e., the fraction of high affinity and/or low receptors in a set, where R^{(k)}=R_{L}^{(k)}+R_{H}^{(k)}; and
determining R^{(k)} for all sets to thereby provide an estimate of the fraction of low affinity and/or high affinity receptors on the surface of the substrate.

2. The method of claim 1 wherein the same aperture, or the same size and shape aperture, is used for all samplings.

4. The method of claim 1 wherein all samplings are averaged.

5. The method of claim 1 wherein the receptors are protein, peptides, antibodies or other antigens.

6. The method of claim 5 wherein the protein, peptides, antibodies or other antigens are present in a membrane fragment or extract.

7. The method of claim 5 wherein the ligands are protein, peptides, antibodies (including polyclonal antibodies, monoclonal antibodies, or fragments thereof) or other antigens.

8. The method of claim 1 wherein the receptors and ligands are oligonucleotides.

9. The method of claim 8 wherein the oligonucleotides are RNA or DNA.

10. A method for determining receptor density (number of receptors, R, per unit area) for receptors having different affinities, where several receptors in a population of receptors are present on the surface of several solid phase carriers and said several receptors bind to the same ligand, and where the receptor-ligand interaction of different receptors has different affinities, K, and wherein the population of receptors includes receptors having a low affinity and receptors having a high affinity, comprising:
contacting, in solution, the solid phase carriers with the ligands under conditions where ligands bind to the receptors and whereby an optical signal of a particular intensity is generated when a ligand is bound to a receptor;
sampling the signal intensities on the carriers using an aperture which admits the optical signal for the sampling;
averaging the signal intensities sampled by the aperture;
dividing, for a particular known ligand concentration in the solution, the averaged signal intensities into a fixed number of segments, wherein each segment includes carriers within a particular range of signal intensities, and repeating this step at several ligand concentrations to generate several sets of segments;
determining the average of the affinity constants for the low affinity and high affinity receptors, respectively designated K_{L} and K_{H}, from the sets of segments;
determining, for a particular set of segments (designated the k-th set), the receptor density of the low affinity receptors, R_{L}(k), and of the high affinity receptors, R_{H}^{(k)};
determining, R_{H}^{(k)}/R^{(k)} and/or R_{L}^{(k)}/R^{(k)}, i.e., the fraction of high affinity and/or low receptors in a set, where R^{(k)}=R_{L}^{(k)}+R_{H}^{(k)}; and
determining R(k) for all sets to thereby provide an estimate of the fraction of low affinity and/or high affinity receptors on the surface of the carriers.

11. The method of claim 10 wherein the aperture accommodates one solid phase carrier.

12. The method of claim 10 wherein the same aperture, or the same size and shape aperture, is used for all samplings.

13. The method of claim 10 wherein all samplings are averaged.

14. The method of claim 10 wherein the receptors are protein, peptides, antibodies or other antigens.

15. The method of claim 14 wherein protein, peptides, antibodies or other antigens are present in a membrane fragment or extract.

16. The method of claim 14 wherein the ligands are protein, peptides, antibodies (including polyclonal antibodies, monoclonal antibodies, or fragments thereof) or other antigens.

17. The method of claim 10 wherein the receptors and ligands are oligonucleotides.

18. The method of claim 17 wherein the oligonucleotides are RNA or DNA.

19. A method of improving the level of confidence of receptor-ligand interaction analysis comprising:
determining receptor density (number of receptors, R, per unit area) for receptors having different affinities, where several receptors in a population of receptors are present on the surface of several solid phase carriers and said several receptors bind to the same ligand, and where the receptor-ligand interaction of different receptors has different affinities, K, and wherein the population of receptors includes receptors having a low affinity and receptors having a high affinity; contacting, in solution, the solid phase carriers with the ligands under conditions where ligands bind to the receptors and whereby an optical signal of a particular intensity is generated when a ligand is bound to a receptor;
sampling the signal intensities on the carriers using an aperture which admits the optical signal for the sampling;
averaging the signal intensities sampled by the aperture;
dividing, for a particular known ligand concentration in the solution, the averaged signal intensities into a fixed number of segments, wherein each segment includes carriers within a particular range of signal intensities, and repeating this step at several ligand concentrations to generate several sets of segments;
determining the average of the affinity constants for the low affinity and high affinity receptors, respectively designated K_{L} and K_{H}, from the sets of segments;
determining, for a particular set of segments (designated the k-th set), the receptor density of the low affinity receptors, R_{L}^{(k)}, and of the high affinity receptors, R_{H}^{(k)};
determining, R_{H}^{(k)}/R^{(k)} and/or R_{L}^{(k)}/R^{(k)} i.e., the fraction of high affinity and/or low receptors in a set, where R^{(k)}=R_{L}^{(k)}+R_{H}^{(k)};
determining R^{(k)} for all sets;
multiplying the number of carriers in each segment by a numerical weight to emphasize R^{H}^{(k)}/R^{(k)} or R_{L}^{(k)}/R^{(k)}, as desired; and
determining the mean and variance of signal intensities for all segments, after multiplying the number of carriers in each segment by the numerical weight.

20. The method of claim 19 wherein the aperture accommodates one solid phase carrier.

21. The method of claim 19 wherein the same aperture, or the same size and shape aperture, is used for all samplings.

22. The method of claim 19 wherein all samplings are averaged.

23. The method of claim 19 wherein the receptors are protein, peptides, antibodies or other antigens.

24. The method of claim 23 wherein protein, peptides, antibodies or other antigens are present in a membrane fragment or extract.

25. The method of claim 19 wherein the ligands are protein, peptides, antibodies (including polyclonal antibodies, monoclonal antibodies, or fragments thereof) or other antigens.

26. The method of claim 19 wherein the receptors and ligands are oligonucleotides.

27. The method of claim 26 wherein the oligonucleotides are RNA or DNA.

28. A method of improving the level of confidence of receptor-ligand interaction analysis comprising:
(i) determining receptor density (number of receptors, R, per unit area) for receptors having different affinities, where several receptors in a population of receptors are present on the surface of several solid phase carriers and said several receptors bind to the same ligand, and where the receptor-ligand interaction of different receptors has different affinities, K, and wherein the population of receptors includes receptors having a low affinity and receptors having a high affinity;
(ii) contacting, in solution, the solid phase carriers with a sample including known ligands under conditions where ligands bind to the receptors and whereby an optical signal of a particular intensity is generated when a ligand is bound to a receptor;
(iii) sampling the signal intensities on the carriers using an aperture which admits the optical signal for the sampling;
(iv) averaging the signal intensities sampled by the aperture;
(v) dividing, for a particular known ligand concentration in the solution, the averaged signal intensities into a fixed number of segments, wherein each segment includes carriers within a particular range of signal intensities, and repeating this step at several ligand concentrations to generate several sets of segments;
(vi) determining the average of the affinity constants for the low affinity and high affinity receptors, respectively designated K_{L} and K_{H}, from the sets of segments;
(vii) determining, for a particular set of segments (designated the k-th set), the receptor density of the low affinity receptors, R_{L}^{(k)}, and of the high affinity receptors, R_{H}^{(k)};
(viii) determining, R_{H}^{(k)}/R^{(k)} and/or R_{L}^{(k)}/R^{(k)}, i.e., the fraction of high affinity and/or low receptors in a set, where R^{(k)}=R_{L}^{(k)}+R_{H}^{(k)};
(ix) determining R(k) for all sets;
(x) multiplying the number of carriers in each segment by a numerical weight to emphasize R_{H}^{(k)}/R^{(k)} or R_{L}^{(k)}/R^{(k)}, as desired;
(xi) determining the mean and variance of signal intensities for all segments, after multiplying the number of carriers in each segment by the numerical weight;
(xii) repeating the steps (i) to (vii) for a different sample;
(xiii) determining the mean and variance of signal intensities for all segments resulting from use of said different sample, after multiplying the number of carriers in each segment by the numerical weight.

29. The method of claim 28 wherein the mean and variance of signal intensities from the sample including known ligands is compared with the mean and variance of signal intensities from the different sample.

30. The method of claim 28 further including transforming R_{H}^{(k)}/R^{(k)} and/or R_{L}^{(k)}/R^{(k)} using a linear function.

31. The method of claim 28 further including transforming R_{H}^{(k)}/R^{(k)} and/or R_{L}^{(k)}/R^{(k)} using a non-linear function.

32. The method of any of claims 10, 9 or 28 wherein the solid phase carriers are microparticles.

33. A method for determining differences in receptor affinity for a ligand, or receptor density (number of receptors, R, per unit area, where the receptors are present on the surface of several solid phase carriers), where the receptor-ligand interaction of different receptors has different affinities, K, and wherein the population of receptors includes receptors having a low affinity and receptors having a high affinity for the ligand, where said differences are determined among two or more different populations of solid phase carriers with the receptors present on their surface, comprising, for each population of solid phase carriers:
determining receptor density for receptors having different affinities; contacting, in solution, the solid phase carriers with the ligands under conditions where ligands bind to the receptors and whereby an optical signal of a particular intensity is generated when a ligand is bound to a receptor;
sampling the signal intensities on the carriers using an aperture which admits the optical signal for the sampling;
averaging the signal intensities sampled by the aperture;
dividing, for a particular known ligand concentration in the solution, the averaged signal intensities into a fixed number of segments, wherein each segment includes carriers within a particular range of signal intensities, and repeating this step at several ligand concentrations to generate several sets of segments;
determining the average of the affinity constants for the low affinity and high affinity receptors, respectively designated K_{L} and K_{H}, from the sets of segments;
determining, for a particular set of segments (designated the k-th set), the receptor density of the low affinity receptors, R_{L}^{(k)}, and of the high affinity receptors, R_{H}^{(k)};
determining, R_{H}^{(k)}/R^{(k)} and/or R_{L}^{(k)}/R^{(k)}, i.e., the fraction of high affinity and/or low receptors in a set, where R^{(k)}=R_{L}^{(k)+R}_{H}^{(k)};
determining R(k) for all sets; and
comparing R_{H}^{(k)}/R^{(k)} and/or Rₗ^{(k)}/R^{(k)} among different populations of solid phase carriers.

34. The method of claim 33 wherein the solid phase carriers are microparticles.

35. The method of claim 33 wherein the aperture accommodates one solid phase carrier.

36. The method of claim 33 wherein the same aperture, or the same size and shape aperture, is used for all samplings.

37. The method of claim 33 wherein all samplings are averaged.

38. The method of claim 33 wherein the receptors are protein, peptides, antibodies or other antigens.

39. The method of claim 38 wherein protein, peptides, antibodies or other antigens are present in a membrane fragment or extract.

40. The method of claim 33 wherein the ligands are protein, peptides, antibodies (including polyclonal antibodies, monoclonal antibodies, or fragments thereof) or other antigens.

41. The method of claim 33 wherein the receptors and ligands are oligonucleotides.

42. The method of claim 41 wherein the oligonucleotides are RNA or DNA.

33. A method for determining differences in affinity of a ligand, or receptor density (number of receptors, R, per unit area, where the receptors are present on the surface of several solid phase carriers), where the receptor-ligand interaction of different ligands has different affinities, K, and wherein a population of ligands includes ligands having a low affinity and ligands having a high affinity for the receptor, where said differences are determined among two or more different populations of ligand molecules, comprising, for each ligand population:
contacting, in solution, the solid phase carriers with the ligands under conditions where ligands bind to the receptors and whereby an optical signal of a particular intensity is generated when a ligand is bound to a receptor;
sampling the signal intensities on the carriers using an aperture which admits the optical signal for the sampling;
averaging the signal intensities sampled by the aperture;
dividing, for a particular known ligand concentration in the solution, the averaged signal intensities into a fixed number of segments, wherein each segment includes carriers within a particular range of signal intensities, and repeating this step at several ligand concentrations to generate several sets of segments;
determining the average of the affinity constants for the low affinity and high affinity ligand types, respectively designated K_{L} and K_{H}, from the sets of segments;
determining, for a particular set of segments (designated the k-th set), the apparent receptor density (i.e., the receptor density calculated based on the assumption that differences in ligands do not affect the affinity of the receptor-ligand interaction) corresponding to receptor-ligand interactions of low affinity, R_{L}^{(k)}, and the apparent receptor density corresponding to receptor-ligand interactions of high affinity, R_{H}^{(k)};
determining, R_{H}^{(k)}/R^{(k)} and/or R_{L}^{(k)}/R^{(k)}, i.e., the fraction of the apparent receptor density respectively corresponding to high affinity receptor-ligand interactions and low affinity receptor-ligand interactions in a set, where R^{(k)}=R_{L}^{(k)}+R_{H}^{(k)};
determining R^{(k)} for all sets; and
comparing R_{H}^{(k)}/R^{(k)} and/or R_{L}^{(k)}/R^{(k)} among said different populations of ligands, where a difference indicates a difference in receptor density or the affinity of the ligands, in a population of ligands, for the receptors.

34. The method of claim 33 wherein the solid phase carriers are microparticles.

35. The method of claim 33 wherein the aperture accommodates one solid phase carrier.

36. The method of claim 33 wherein the same aperture, or the same size and shape aperture, is used for all samplings.

37. The method of claim 33 wherein all samplings are averaged.

38. The method of claim 33 wherein the receptors are protein, peptides, antibodies or other antigens.

39. The method of claim 38 wherein protein, peptides, antibodies or other antigens are present in a membrane fragment or extract.

40. The method of claim 33 wherein the ligands are protein, peptides, antibodies (including polyclonal antibodies, monoclonal antibodies, or fragments thereof) or other antigens.

41. The method of claim 33 wherein the receptors and ligands are oligonucleotides.

42. The method of claim 41 wherein the oligonucleotides are RNA or DNA.

43. A method for determining heterogeneity of a first solution including ligands which are capable of binding to the same receptor, comprising:
determining apparent receptor density (number of receptors, R, per unit area) for ligands of a specified affinity in a population of ligands in a solution, several types of ligands in said population being capable of binding to the receptors, and where the receptor- ligand interaction can have different affinities, K, and wherein the population includes ligands having a low affinity and ligands having a high affinity for the receptors, by:
(i) contacting, in solution, the solid phase carriers with the ligands under conditions where the ligands bind to the receptors and whereby an optical signal of a particular intensity is generated when a ligand is bound to a receptor;
(ii) sampling the signal intensities on the surfaces using an aperture which admits the optical signal for the sampling;
(iii) averaging the signal intensities sampled by the aperture;
(iv) dividing, for a particular known ligand concentration in the solution, the averaged signal intensities into a fixed number of segments, wherein each segment includes samplings within a particular range of signal intensities, and repeating this step at several ligand concentrations to generate several sets of segments;
(v) determining the average of the affinity constants for the low affinity and high affinity ligand types, respectively designated K_{L} and K_{H}, from the sets of segments;
(vi) determining, for a particular set of segments (designated the k-th set), the apparent receptor density (i.e., the receptor density calculated based on the assumption that differences in ligands do not affect the affinity of the receptor-ligand interaction) corresponding to receptor-ligand interactions of low affinity, R_{L}^{(k)}, and the apparent receptor density corresponding to receptor-ligand interactions of high affinity, RH(k);
(vii) determining, R_{H}^{(k)}/R^{(k)} and/or R_{L}^{(k)}/R^{(k)}, i.e., the fraction of apparent receptor density respectively corresponding to reflecting high affinity receptor-ligand interactions and low affinity receptor-ligand interactions in a set, where R^{(k)}=R_{L}^{(k)}+R_{H}^{(k)};
(viii) determining R^{(k)} for all sets; and
(ix) performing the steps (i) to (viii) above for a set of reference solutions containing a known concentration of a known ligand, and for a set of solutions containing a known concentration of the first solution, and comparing R_{H}^{(k)}/R^{(k)} and/or R_{L}^{(k)}/R^{(k)} for the set of reference solutions and for the first solution, a difference in the results indicating heterogeneity in the ligands in the first solution.

44. The method of claim 43 wherein the solid phase carriers are microparticles.

45. The method of claim 43 wherein the aperture accommodates one solid phase carrier.

46. The method of claim 43 wherein the same aperture, or the same size and shape aperture, is used for all samplings.

47. The method of claim 43 wherein all samplings are averaged.

48. The method of claim 43 wherein the receptors are protein, peptides, antibodies or other antigens.

49. The method of claim 48 wherein protein, peptides, antibodies or other antigens are present in a membrane fragment or extract.

50. The method of claim 43 wherein the ligands are protein, peptides, antibodies (including polyclonal antibodies, monoclonal antibodies, or fragments thereof) or other antigens.

51. The method of claim 43 wherein the receptors and ligands are oligonucleotides.

52. The method of claim 51 wherein the oligonucleotides are RNA or DNA.
